SEQUENCE LISTING (12 sequences)

SEQ ID NO: 1 - DNA, 2798 bp, Homo sapiens
gggtcgtgagaaaatacgcaggaagagataccccgtggacatgtttcccttggaaagtggagggaaccctctgagccgtgattccgcctgaattaagcagtggcgggcactggcctggcaggcggcttctactgccttggatttgcacctctaaggggtcacaggggccacccccgaaacaagacaaagtgtgatcatggacaggccctcggggggccggaagaaggaacggctccagtgatgaaagctgaacgccagctctctccccatcgttctcccggaccaggagagggctgctcggcagggctaggtttaaaaaccttccatgcaacttacgaaggcatccacagtgacttctgctgctgtggtacgttctgccctcccctcctccccgccccatcctgttctcggctacaagttatttatatgtacagtgtcatctgaacgtgcacgggcctatcccaggacatcacacacaaagaggcggcggtgaacccaagccgccgggccggcctgtggctcagtgtgtgtttcagaagtcccggccccaggcagctctcctgaacagagccactggaactcaggagggtcaggaccacagcccaaacaactgcgaccgaaaacaccctacagggcttgagaactaaaaaataaggagggatctgggcagccgagctgcctcctaaagaagccacgtcatagagaaaactgcttgctctatccatgagacggaggcggccggggtgtcctgccagcagacacgggtctgtccatcaccggccaccacgctgcctctgtccttcactcccatttaagggaaaacgatatctcttcatctttctgccctggacattggaaatcagcggtgagaacagtgctcttgctacaaaatgctcaggttctagaatgctcccatctttccttccattccacatggagtttatccaaagcacaaaggccagtcacggcactgacatccgtggtgcccgctcaccggggagtgggaagcacaagcctgtcctcagaggaccgtgactgtgagcccgcagctgcttgtgggtggggccagcccgcagctcctcctggaggcctcgggcttcttgctgctgcatttctgtgacatgtggagggaggatcgttttgacacctgcctgggaagtttcaaaggagatggcctctgggcaggggttttaaactgaagggcaccacacagatccagatgtgaactgtggctgttgtttgcaattgaagcagagatgcagaggatggaacaggagtgctggccatgctgctctatggccttggcccagccaagacctgcagtcaatggggctacgggacccccgctacacagggacctgggaaccacctttgccgcggttgaccgctgggcccgccatgcaccgcccacacctggctgtgcctggagactgagagcagggggtgacggtgggcctggaagagtctctcccttttgcaaacgccagccacagaggtttatttttctccctgagagaaggaagagtgaaggctgagactgagacagaaggacagaaggagaagacttagaagccagtgagcccggaaccacgagacgcgctctgaggacacggggcctcggggaaaccgggcacgggcaggggttggtcttggcttccccgcaacattgccgcgagctcgggtgccgtctccactcgcaggctggcctgtcctctgcagccacgagctcaggcaccgtctccgttcataggccagcctgttctctgcagcctgaggtccctgtggtgccaacatccagatgctgagataattctgccagggtgttggctgatcggtgattggtggttggctgttccgtgagttctgcattgatcttgctggctgagcgtccaatgcggaggggcaggtgctgttagggtctccactcccatccctctccccaggactcatcacccactgcaaccgtaagctgctaggggctcacagccgtgagtctttgggacgggtggtcccacctggcatgtagctcaggctcctagccccaggaccctgccgctgctcccctcccgcccacatcttggcttagccgtcttccctggctgccagccccttcccctggtcttagttggcttgagccactaaactgaaatagcacaccctgaggccttcaacagcagacctgatttctcaccatctggaggctggaggtccaggatccaggcccccaaggctgctttctcccccactcccccacccaccttccaggacacgcagactccaccctcttgctgccctgtctcagggctgtccctctgggcaagcaggcccctggcatccccgtgtgtcttctcctcctgttcttataaggacaccagtcatactggatctgggcccaccctagtgacctcatttaccttaatcacctctgaaagaccttgtctcaaaaacagccacattctggggcactgagggttagggctttaacatgggaatttgagggatgcaattcaccccataaaacccagaccattcccccagtaaatcactttcacaagagctcttctctcaggctttgcttctgggggatcccccttgagatggatccatctagcttcctgcttctcaaagaacagaggggagagctctggtgcgaagggaacgggtggcctcggggacctgcggataagctgctggcactgccaggggacaggctgagctgaaagccaggaggagggcggcgaggcccggggccggacctcattcttctgagtgttataaaatctggctttgctcagaaa

SEQ ID NO: 2 - DNA, 15 bp, Homo sapiens
agaggtgattaaggt

SEQ ID NO: 3 - DNA, 16 bp, Homo sapiens
agtgaaggacagaggc

SEQ ID NO: 4 - DNA, 15 bp, Homo sapiens
aacacgtctatacgc

SEQ ID NO: 5 - DNA, 20 bp, Homo sapiens
cacctgcctgggaagtttca

SEQ ID NO: 6 - DNA, 20 bp, Homo sapiens
atctggatctgtgtggtgcc

SEQ ID NO: 7 - DNA, 21 bp, Mus musculus
aataaagcagtgggtgctggg

SEQ ID NO: 8 - DNA, 23 bp, Mus musculus
actgttgcaacccatttatctga

SEQ ID NO: 9 - DNA, 16 bp, Mus musculus
ggcacacgaacagctg

SEQ ID NO: 10 - DNA, 16 bp, Mus musculus
tggcacacgaacagct

SEQ ID NO: 11 - DNA, 16 bp, Mus musculus
tgtctgcacttaatta

SEQ ID NO: 12 - DNA, 16 bp, Mus musculus
gtctgcacttaattaa

(19) United States
(12) Patent Application Publication
(10) Pub. No.: US 2018/0044672 A1
(43) Pub. Date: Feb. 15, 2018

(54) Pericyte Long Non-Coding RNAs

(71) Applicant: Johann Wolfgang Goethe-Universität Frankfurt am Main, Frankfurt am Main (DE)

(72) Inventors: Christoph Michael Zehendner, Mainz (DE); Stefanie Dimmeler, Frankfurt (DE); Andreas Zeiher, Frankfurt (DE)

(21) Appl. No.: 15/554,391

(22) PCT Filed: Mar. 18, 2016
(86) PCT No.: PCT/EP2016/056013

§ 371 Date: Aug. 29, 2017

(30) Foreign Application Priority Data
Mar. 20, 2015 (DE) 10 2015 003 656.4

(51) Int. Cl. C12N 15/113; C12Q 1/68; A61K 31/7088

(52) U.S. Cl. CPC C12N 15/113; A61K 31/7088; C12Q 1/68; C12N 2310/113; C12N 2320/31

(57) ABSTRACT

The present invention provides novel non-coding RNAs (lncRNA) that were identified to be expressed in pericytes upon hypoxia. The lncRNA of the invention positively affect Platelet-derived Growth Factor Receptor (PDGFR) beta expression, pericytes proliferation and pericyte recruitment to endothelial cells. The invention provides inhibitors of the lncRNA for use in the treatment of diseases mediated by PDGFR expression. For example the invention described antisense approaches to target the lncRNA of the invention. Furthermore, the invention provides lncRNA inhibitors as amplifier of therapeutic PDGFR inhibitors such as imatinib or other tyrosine kinase inhibitors. lncRNA inhibitors and methods for screening modulators of lncRNA expression and/or function are provided.

### FIELD OF THE INVENTION

The present invention provides novel non-coding RNAs (lncRNA) that were identified to be expressed in pericytes upon hypoxia. The lncRNA of the invention positively affect Platelet-derived Growth Factor Receptor (PDGFR) beta expression, pericytes proliferation and pericyte recruitment to endothelial cells. The invention provides inhibitors of the lncRNA for use in the treatment of diseases mediated by PDGFR expression. For example the invention described antisense approaches to target the lncRNA of the invention. Furthermore, the invention provides lncRNA inhibitors as amplifier of therapeutic PDGFR inhibitors such as imatinib or other tyrosine kinase inhibitors. lncRNA inhibitors and methods for screening modulators of lncRNA expression and/or function are provided.

### DESCRIPTION

Pericytes (PC) are abundantly expressed perivascular cells that essentially contribute to proper function of heart, brain, lungs, and kidneys. Moreover, PC stabilize tumor vascularization in various malignant processes. It is well documented that tyrosine kinase signaling through PDGFRβ crucially regulates PC survival, proliferation and PC-endothelial interactions. Platelet-derived growth factors (PDGFs) are potent mitogens that exist as five different dimeric configurations composed of four different isoform subunits: A, B, C and D. The five dimeric forms of the PDGFs are AA, BB, AB, CC and DD, which are formed by disulfide linkage of the corresponding individual PDGF monomers.

PDGF ligands exert their biological effects through their interactions with PDGF receptors (PDGFRs). PDGFRs are single-pass, transmembrane, tyrosine kinase receptors composed of heterodimeric or homodimeric associations of an alpha (α) receptor chain (PDGFR-alpha) and/or a beta (β) receptor chain (PDGFR-beta). Thus, active PDGFRs may consist of αα, ββ or αβ receptor chain pairings. PDGFRs share a common domain structure, including five extracellular immunoglobulin (Ig) loops, a transmembrane domain, and a split intracellular tyrosine kinase (TK) domain. The interaction between dimeric PDGF ligands and PDGFRs leads to receptor chain dimerization, receptor autophosphorylation and intracellular signal transduction. It has been demonstrated in vitro that ββ receptors are activated by PDGF-BB and -DD, while αβ receptors are activated by PDGF-BB, -CC, -DD and -AB, and αα receptors are activated by PDGF-AA, -BB, -CC and -AB (see Andrae et al. (2008) Genes Dev 22(10):1276-1312).

PDGF signaling has been implicated in various human diseases including diseases associated with pathological neovascularization, vascular and fibrotic diseases, tumor growth and eye diseases. Accordingly, inhibitors of PDGF signaling have been suggested for use in a variety of therapeutic settings. For example, inhibitors of PDGFR-beta have been proposed for use in treating various diseases and disorders. (Andrae et al. (2008) Genes Dev 22(10):1276-1312). PDGFR-beta inhibitors include non-specific small molecule tyrosine kinase inhibitors such as imatinib mesylate, sunitinib malate and CP-673451, as well as anti-PDGFR-beta antibodies (see, e.g., U.S. Pat. Nos. 7,060,271; 5,882,644; 7,740,850; and U.S. Patent Appl. Publ. No. 2011/0177074). Anti-ligand aptamers (e.g., anti-PDGF-B) have also been proposed for therapeutic applications. Nonetheless, a need exists in the art for new, highly specific and potent inhibitors of PDGF signaling.

RNA sequencing revealed that the majority of the genome is transcribed, however, most transcripts do not encode for proteins. According to their size, these so called “non-coding RNAs” are divided in small non-coding RNAs (<200 nucleotides) and long non-coding RNAs (lncRNAs; >200 nt) such as natural antisense transcripts (NATs), long intergenic non-coding RNAs (lincRNAs) and circular RNAs. Whereas the function and mechanism of distinct noncoding RNA species is well understood and clearly defined (e.g. miRNAs), lncRNAs exhibit various molecular functions, for example by acting as scaffold or guide for proteins/RNAs or as molecular sponges. Therefore, lncRNAs can interfere with gene expression and signaling pathways at various stages. Specifically, lncRNAs were shown to recruit chromatin modifying enzymes, to act as decoys for RNA and protein binding partners, and to modulate splicing and mRNA degradation. Whereas microRNAs are well established regulators of endothelial cell function, vessel growth and remodeling, the regulation and function of lncRNAs in the endothelium is poorly understood.

Long ncRNAs vary in length from several hundred bases to tens of kilobases and may be located separate from protein coding genes (long intergenic ncRNAs or lincRNAs), or reside near or within protein coding genes (Guttman et al. (2009) Nature 458:223-227; Katayama et al. (2005) Science 309:1564-1566). Recent evidence indicates that active enhancer elements may also be transcribed as lncRNAs (Kim et al. (2010) Nature 465:182-187; De Santa et al. (2010) PLoS Biol. 8:e1000384).

Several lncRNAs have been implicated in transcriptional regulation. For example, in the CCND1 (encoding cyclin Dl) promoter, an ncRNA transcribed 2 kb upstream of CCND1 is induced by ionizing radiation and regulates transcription of CCND1 in cis by forming a ribonucleoprotein repressor complex (Wang et al. (2008) Nature 454:126-130). This ncRNA binds to and allosterically activates the RNA-binding protein TLS (translated in liposarcoma), which inhibits histone acetyltransferases, resulting in repression of CCND1 transcription. Another example is the antisense ncRNA CDKN2B-AS1 (also known as p15AS or ANRIL), which overlaps the p15 coding sequence. Expression of CDKN2B-AS is increased in human leukemias and inversely correlated with p15 expression (Pasmant et al. (2007) Cancer Res. 67:3963-3969; Yu et al. (2008) Nature 451:202-206). CDKN2B-AS1 can transcriptionally silence p15 directly as well as through induction of heterochromatin formation. Many well-studied lncRNAs, such as those involved in dosage compensation and imprinting, regulate gene expression in cis (Lee (2009) Genes Dev. 23:1831-1842). Other lncRNAs, such as HOTAIR and linc-p21 regulate the activity of distantly located genes in trans (Rinn et al. (2007) Cell 129:1311-1323; Gupta et al. (2010) Nature 464:1071-1076; and Huarte et al. (2010) Cell 142:409-419).

In view of the state of the art it was therefore an object of the present invention to provide novel options for the treatment of diseases that are associated with pericyte function and/or PDGFR signaling, such as angiogenesis, breakdown of endothelial barrier function in stroke malignancy or inflammation or cardiovascular disorders, specifically cancers such as leukemia. The present invention seeks to provide new drug targets for these diseases based on comprehensive deep sequencing approaches of the pericyte transcriptome in response to hypoxia.

The above problem is solved in a first aspect by 1. An inhibitor of a long non-coding RNA (lncRNA), the lncRNA selected from TYKRIL (also known as AP001046.5), MIR210HG, RP11-367F23.1, H19, RP11-44N21.1, AC006273.7, RP11-120D5.1, RP11-443B7.1, AC005082.12, RP11-65J21.3, or AC008746.12 for use in the treatment of a disease.

In context of the present disclosure TYKRIL (also known as AP001046.5) is a long noncoding RNA (lncRNA) comprising a sequence having at least 60%, 70%, 80%, 85%, 90%, 95%, 96%, 97%, 98%, 99% or 100% sequence identity to SEQ ID NO: 1

The other lncRNA are found in the human genome at the following positions:

TABLE 1
lncRNA Sequences (hg19)

| Ensembl version | Name | Locus (chr: position) |
|---|---|---|
| ENSG00000247095 | MIR210HG | 11: 565659-568457 |
| ENSG00000228216 | RP11-367F23.1 | 9: 93719541-93727675 |
| ENSG00000130600 | H19 | 11: 2016405-2022700 |
| ENSG00000257556 | RP11-44N21.1 | 14: 105559945-105565341 |
| ENSG00000266927 | AC006273.7 | 19: 786364-786965 |
| ENSG00000234129 | RP11-120D5.1 | X: 10981959-11129258 |
| ENSG00000237989 | AP001046.5 | 21: 44778026-44782229 |
| ENSG00000238005 | RP11-443B7.1 | 1: 235092977-235105809 |
| ENSG00000226816 | AC005082.12 | 7: 23245631-23247664 |
| ENSG00000262454 | RP11-65J21.3 | 16: 14396144-14420210 |
| ENSG00000267838 | AC008746.12 | 19: 54949846-54950362 |

MIR210HG is a long noncoding RNA (lncRNA) comprising a sequence having at least 80% sequence identity to the above chromosomal location sequence, RP11-367F23.1 is a long noncoding RNA (lncRNA) comprising a sequence having at least 80% sequence identity to the above chromosomal location sequence, H19, is a long noncoding RNA (lncRNA) comprising a sequence having at least 80% sequence identity to the above chromosomal location sequence, RP11-44N21.1 is a long noncoding RNA (lncRNA) comprising a sequence having at least 80% sequence identity to the above chromosomal location sequence, AC006273.7 is a long noncoding RNA (lncRNA) comprising a sequence having at least 80% sequence identity to the above chromosomal location sequence, RP11-120D5.1 is a long noncoding RNA (lncRNA) comprising a sequence having at least 80% sequence identity to the above chromosomal location sequence, RP11-443B7.1 is a long noncoding RNA (lncRNA) comprising a sequence having at least 80% sequence identity to the above chromosomal location sequence, AC005082.12 is a long noncoding RNA (lncRNA) comprising a sequence having at least 80% sequence identity to the above chromosomal location sequence, RP11-65J21.3 (also known as HypERrinc) is a long noncoding RNA (lncRNA) comprising a sequence having at least 80% sequence identity to the above chromosomal location sequence, or AC008746.12 is a long noncoding RNA (lncRNA) comprising a sequence having at least 80% sequence identity to the above chromosomal location sequence.

The present invention is based on RNA deep sequencing (RNA seq) identified hypoxia induced lncRNAs. In precisely controlled in vitro assays, it is shown that the hypoxia induced lncRNA TYKRIL (Tyrosine Kinase Receptor Inducing lncRNA, also known as AP001046.5) is a major regulator of PDGFRβ expression in human pericytes. Knockdown of TYKRIL with locked nucleid acid Gapmers causes a significant downregulation of PDGFRβ on mRNA and protein level. In addition, TYKRIL silencing impairs pericyte proliferation and differentiation. Moreover, TYKRIL deficiency results in failure of pericyte recruitment towards endothelial cells. This disclosure thus indicates that TYKRIL, and the other identified hypoxia regulated lncRNAs are essential for pericyte function and represent a novel target for the modulation of PDGFRβ expression in health and disease.

The following specific embodiments of the invention described in context of the present disclosure shall be understood to refer to all lncRNA molecules of the invention as disclosed herein. However, particular emphasis is put on embodiments relating to the lncRNA TYKRIL as drug target in medical applications. Hence, all embodiments relating to TYKRIL agonists or inhibitors as lncRNA inhibitors or agonists, or methods for screening such compounds, are preferred solutions to the problems in the prior art provided by the present invention.

Also it is disclosed that lncRNA inhibitors are a preferred embodiment of the invention.

The present invention preferably provides as inhibitor an inhibitor of lncRNA expression and/or function. Preferred embodiments of the invention provide as inhibitors an lncRNA antisense molecule, such as antisense RNA, RNA interference (RNAi), siRNA, esiRNA, shRNA, miRNA, decoys, RNA aptamers, GapmeRs, LNA molecules; or an antisense expression molecule, or small molecule inhibitors, RNA/DNA-binding proteins/peptides, or an anti-lncRNA antibody. A detailed description of lncRNA antagonists or inhibitors is further provided below.

The lncRNA antisense molecule more preferably is a nucleic acid oligomer having a contiguous nucleotide sequence of a total of 8 to 100 nucleotides, wherein said contiguous nucleotide sequence is at least 80% identical to the reverse complement of the sequence of the lncRNA. In preferred embodiments the lncRNA antisense molecule is a TYKRIL antisense molecule.

An antisense molecule of the invention may be a nucleic acid oligomer having a contiguous nucleotide sequence of 8 to 100 nucleotides, preferably 8 to 50, 8 to 40, 8 to 30, 8 to 20, or 9 to 100, 9 to 50, 9 to 40, 9 to 30, 9 to 20, or 10 to 100, 10 to 50, 10 to 40, 10 to 30, 10 to 20, nucleotides. Most preferred are oligomers with 10 to 30 nucleotides.

As also described in detail herein below, the antisense molecule may comprise a contiguous nucleotide sequence having at least one nucleic acid modification. The at least one nucleic acid modification is preferably selected from 2′-O-alkyl modifications, such as 2′-O-methoxyethyl (MOE) or 2′-O-Methyl (OMe), ethylene-bridged nucleic acids (ENA), peptide nucleic acid (PNA), 2′-fluoro (2′-F) nucleic acids such as 2′-fluoro N3-P5′-phosphoramidites, 1′, 5′-anhydrohexitol nucleic acids (HNAs), and locked nucleic acid (LNA).

As mentioned above, particular preferred is that the lncRNA inhibitor of the invention is an inhibitor of TYKRIL expression and/or function. In this regard the disclosure provides as preferred molecule an antisense molecule comprising a contiguous nucleotide sequence having least 80%, 85%, 90%, 95%, 96%, 97%, 98%, 99% or 100% sequence identity to SEQ ID NO: 2 or 3, preferably wherein the antisense molecule is an LNA GapmeR.

The present invention is based on the development of lncRNA as drug targets. Therefore, a central aspect of the invention pertains to the modulation of the expression or function of the lncRNAs as disclosed herein, preferably in the context of a medical treatment. For means or compounds enhancing the expression or function of a lncRNA the present invention will refer to such means or compounds as “agonists” of a respective lncRNA. Alternatively, the expression and/or function of an lncRNA may be reduced or inhibited. In this case the present invention will refer to these mediators of the effect as “inhibitor” or “antagonist” of the respective lncRNA of the invention. Since lncRNA are RNA-molecules which mediate their biological activity either in the cellular cytoplasm or in the cell nucleus the person of skill can harness all known methods that intervene with the natural RNA metabolism.

In some embodiments an agonist of a lncRNA of the invention is selected from an lncRNA molecule of the invention or a homolog thereof. The person of skill will appreciate that the herein explained function and effects of lncRNA agonists are reversed or contrary to those functions and effect disclosed for the lncRNA inhibitors. An lncRNA molecule is an RNA molecule corresponding to a lncRNA sequence as disclosed herein above (the lncRNA sequences). A homolog in the context of the invention is a nucleic acid, preferably an RNA molecule, which is homologous to any of the lncRNA of the herein described invention, and preferably comprises a sequence of at least 60% sequence identity to any one of the lncRNA sequences as defined herein above (lncRNA sequences). Further preferred homologs of the invention comprise a nucleic acid sequence that is at least 70%, 80%, 90%, 95%, 96%, 97%, 98%, or most preferably 99% identical to an lncRNA sequence as defined herein above.

Alternatively, the present invention provides expression constructs of lncRNAs as agonists of the invention. An lncRNA expression construct of the invention comprises preferably an expressible sequence of lncRNA of the invention, optionally of homologs thereof, operatively linked to a promoter sequence. Since expression constructs may be used both to express an agonist or an antagonist of an lncRNA of the invention a detailed description of expression constructs is provided herein below.

In order to impair lncRNA expression/function in accordance with the herein described invention the person of skill may choose any suitable methodology for inhibiting RNA expression. In particular preferred are antisense approaches, which apply sequence complementary nucleic acid polymers (antagonists/inhibitors) which mediate the inhibition or destruction of a target RNAs and thereby impair lncRNA function.

According to some embodiments, an lncRNA of the invention may be targeted using an inhibiting agent or therapeutic—an antagonist of the lncRNA—targeting strategy such as antisense RNA, RNA interference (RNAi), siRNA, esiRNA, shRNA, miRNA, decoys, RNA aptamers, small molecule inhibitors, RNA/DNA-binding proteins/peptides, GapmeRs, LNA molecules or other compounds with different formulations to inhibit one or more physiological actions effected by lncRNA. The antisense antagonists of the invention may either be directly administered or used, or alternatively, may be expressed using an expression construct, for example a construct expressing a miRNA having a sequence complementary to an lncRNA of the invention.

For the inhibition of the lncRNA of the invention, it is in certain embodiments preferred to use antisense oligonucleotides in order to impair the lncRNA expression or function. Antisense oligonucleotides (ASOs) or oligomers (the terms may be used interchanging) are synthetic nucleic acids that bind to a complementary target and suppress function of that target. Typically ASOs are used to reduce or alter expression of RNA targets—lncRNA is one preferred example of an RNA that can be targeted by ASOs. As a general principle, ASOs can suppress RNA function via two different mechanisms of action: 1) by steric blocking, wherein the ASO tightly binds the target nucleic acid and inactivates that species, preventing its participation in cellular activities, or 2) by triggering degradation, wherein the ASO binds the target and leads to activation of a cellular nuclease that degrades the targeted nucleic acid species. One class of “target degrading”. ASOs may be composed of several types of nucleic acids, such as RNA, DNA or PNA.

In order to enhance the half-life of an ASO, modifications of the nucleic acids can be introduced. It is in particular useful to protect the ASO from degradation by cellular endonucleases. One modification that gained widespread use comprised DNA modified with phosphorothioate groups (PS). PS modification of internucleotide linkages confers nuclease resistance, making the ASOs more stable both in serum and in cells. As an added benefit, the PS modification also increases binding of the ASO to serum proteins, such as albumin, which decreases the rate of renal excretion following intravenous injection, thereby improving pharmacokinetics and improving functional performance. Therefore, PS modified ASOs are encompassed by the present invention.

Further modifications target the 3 ‘-end of an ASO molecule, for example “Gapmer” compounds having 2’-methoxyethylriboses (MOE's) providing 2′-modified “wings” at the 3′ and 5′ ends flanking a central 2′-deoxy gap region. ASO modifications that improve both binding affinity and nuclease resistance typically are modified nucleosides including locked nucleic acids (LNA), wherein a methyl bridge connects the 2′-oxygen and the 4′-carbon, locking the ribose in an A-form conformation; variations of LNA are also available, such as ethylene-bridged nucleic acids (ENA) that contain an additional methyl group, amino-LNA and thio-LNA. Additionally, other 2′-modifications, such as 2′-0-methoxyethyl (MOE) or 2′-fluoro (2′-F), can also be incorporated into ASOs. Such exemplary modifications are comprised by the present invention.

In the context of the present invention this means that the term “antisense oligonucleotide” pertains to a nucleotide sequence that is complementary to at least a portion of a target lncRNA sequence of the invention. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases as already mentioned above. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides. PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures. Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on a given lncRNA sequence such as those provided herein. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, siRNA technology may be applied to inhibit expression of a lncRNA of the invention. Application of nucleic acid fragments such as siRNA fragments that correspond with regions in lncRNA gene and which selectively target a lncRNA may be used to block lncRNA expression or function.

SiRNA, small interfering RNA molecules, corresponding to a region in the lncRNA sequence are made using well-established methods of nucleic acid syntheses as outlined above with respect to antisense oligonucleotides. Since the structure of target lnc-RNAs is known, fragments of RNA/DNA that correspond therewith can readily be made. The effectiveness of selected siRNA to impair lncRNA function or expression, for example via targeted degradation, can be confirmed using a lncRNA-expressing cell line. Briefly, selected siRNA may be incubated with a lncRNA-expressing cell line under appropriate growth conditions. Following a sufficient reaction time, i.e. for the siRNA to bind with lncRNA to result in decreased levels of the lnc-RNA, the reaction mixture is tested to determine if such a decrease has occurred, for example via quantitative PCR, northern blotting etc.

Antisense oligonucleotides in accordance with the invention may comprise at least one modification that is incorporated at the terminal end of an antisense oligonucleotide, or between two bases of the antisense oligonucleotide, wherein the modification increases binding affinity and nuclease resistance of the antisense oligonucleotide. In one embodiment, the antisense oligonucleotide comprises at least one modification that is located within three bases of a terminal nucleotide. In another embodiment, the antisense oligonucleotide comprises at least one modification that is located between a terminal base and a penultimate base of either the 3′- or the 5′-end of the oligonucleotide. In another embodiment, the antisense oligonucleotide comprises a modification at a terminal end of the oligonucleotide. In a further embodiment, the antisense oligonucleotide comprises a modification at the terminal end or between the terminal base and the penultimate base of both the 3′- and the 5′-ends of the antisense oligonucleotide. In yet a further embodiment, the oligonucleotide contains a non-base modifier at a terminal end or between the terminal base and the penultimate base at the 5 ‘-end and at the 3’-end.

Also comprised are antibodies binding to an inhibiting a lncRNA of the invention.

The lncRNA inhibitors, or also agonists, are specifically useful in medicine as therapeutics for the treatment of a disease. A disease in preferred embodiments of the present invention may be a disease associated with an increased expression of a Platelet-derived growth factor receptor (PDGFR) and/or associated with an increased or decreased expression/function of p53, preferably PDGFR-β. Such diseases may be selected from a disease associated with pathological angiogenesis, such as fibrotic disease (fibrosis), cardiovascular disease, pulmonary and/or a tumorous disease (cancer).

The term “pathological angiogenesis” refers to the excessive formation and growth of blood vessels during the maintenance and the progression of several disease states. Examples where pathological angiogenesis can occur are blood vessels (atherosclerosis, hemangioma, hemangioendothelioma), bone and joints (rheumatoid arthritis, synovitis, bone and cartilage destruction, osteomyelitis, pannus growth, osteophyte formation, neoplasms and metastasis), skin (warts, pyogenic granulomas, hair growth, Kaposi's sarcoma, scar keloids, allergic oedema, neoplasms), liver, kidney, lung, ear and other epithelia (inflammatory and infectious processes (including hepatitis, glomerulonephritis, pneumonia), asthma, nasal polyps, otitis, transplantation, liver regeneration, neoplasms and metastasis), uterus, ovary and placenta (dysfunctional uterine bleeding (due to intrauterine contraceptive devices), follicular cyst formation, ovarian hyperstimulation syndrome, endometriosis, neoplasms), brain, nerves and eye (retinopathy of prematurity, diabetic retinopathy, choroidal and other intraocular disorders, leukomalacia, neoplasms and metastasis), heart and skeletal muscle due to work overload, adipose tissue (obesity), endocrine organs (thyroiditis, thyroid enlargement, pancreas transplantation), hematopoiesis (AIDS (Kaposi), hematologic malignancies (leukemias, etc.), tumour induced new blood vessels. The pathological angiogenesis may occur in connection with a proliferative disorder, most preferably in connection with a cancer disease. A cancer may be selected from the group consisting of liver cancer, brain tumors in particular glioblastoma, lung cancer, breast cancer, colorectal cancer, stomach cancer and melanoma, most preferably where-in the cancer is solid cancer, even more preferably a metastatic solid cancer.

Leukemia is a preferred cancer of the invention. The term leukemia as used herein includes, but is not limited to, chronic myelogenous leukaemia (CML) and acute lymphocyte leukaemia (ALL), especially Philadelphia-chromosome positive acute lymphocyte leukaemia (Ph+ALL). Preferably, the variant of leukaemia to be treated by the methods disclosed herein is CML, in particular drug resistant CML, such as imatinib resistant leukemia.

Another preferred disease is glioblastoma, a primary brain tumor involving glial cells.

Moreover, pulmonary arterial hypertension (PAH), a disease with elevated artery pressure in the pulmonary system, is a preferred disease.

In another embodiment of the invention the “pathological angiogenesis” is a cancer disease or cardiopulmonary disease associated with a reduced expression or altered function or mutation of p53. Since the present invention provides inhibitors that upregulate p53 and enhance the binding of its co-activator p300 on p53, the compounds of the invention are generally useful for the treatment of cancer or adverse organ remodeling and tissue scarring.

A cardiovascular disease in context of the present invention may be a disease associated with a pathological repressed endothelial cell repair, cell growth and/or cell division or is a disease treatable by improving endothelial cell repair, cell growth and/or cell division. Generally, the term “cardiovascular disease,” as used herein, is intended to refer to all pathological states leading to a narrowing and/or occlusion of blood vessels throughout the body. In particular, the term “cardiovascular disease” refers to conditions including atherosclerosis, thrombosis and other related pathological states, especially within arteries of the heart and brain. Accordingly, the term “cardiovascular disease” encompasses, without limitation, various types of heart disease, as well as Alzheimer's disease and vascular dimension.

In preferred embodiments of the invention the cardiovascular disease is selected from the group consisting of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerot-ic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric is-chemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, frostbite, graft-versus-host dis-ease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosar-coma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular dis-ease of the kidney, vascular inflammatory conditions, von Hippel-Lindau syndrome, or wounds to tissues or organs. The inhibitors of the present invention are useful to prevent organ remodeling in context of the aforementioned cardiovascular diseases. One preferred disease is also pulmonary arterial hypertension (PAH).

Exemplary fibrotic diseases that are treatable by administering the inhibitors of the invention include pulmonary fibrosis (e.g., idiopathic pulmonary fibrosis, bleomycin-induced pulmonary fibrosis, asbestos-induced pulmonary fibrosis, and bronchiolitis obliterans syndrome), chronic asthma, fibrosis associated with acute lung injury and acute respiratory distress (e.g., bacterial pneumonia induced fibrosis, trauma induced fibrosis, viral pneumonia induced fibrosis, ventilator induced fibrosis, non-pulmonary sepsis induced fibrosis and aspiration induced fibrosis), silicosis, radiation-induced fibrosis, chronic obstructive pulmonary disease (COPD), ocular fibrosis (e.g., ocular fibrotic scarring), skin fibrosis (e.g., scleroderma), hepatic fibrosis (e.g., cirrhosis, alcohol-induced liver fibrosis, non-alcoholic steatohepatitis (NASH), bilary duct injury, primary bilary cirrhosis, infection- or viral-induced liver fibrosis [e.g., chronic HCV infection], autoimmune hepatitis), kidney (renal) fibrosis, cardiac fibrosis, atherosclerosis, stent restenosis, and myelofibrosis.

Other preferred embodiments of the invention relates to the medical use of the herein disclosed inhibitors of lncRNA—in particular TYKRIL inhibitors—wherein the treatment comprises the simultaneous or sequential administration of the inhibitor of the lncRNA and a second therapeutic agent, such as a PDGFR-inhibitor. The PDGFR-inhibitor is preferably an anti-PDGFR-antibody, a small molecule tyrosine kinase inhibitor, such as imatinib (preferred), sorafenib, lapatinib, BIRB-796 and AZD-1152; AMG706, Zactima (ZD6474), MP-412, sorafenib (BAY 43-9006), dasatinib, CEP-701 (lestaurtinib), XL647, XL999, Tykerb (lapatinib), MLN518, PKC412, STI571, AEE 788, OSI-930, OSI-817, Sutent (sunitinib maleate), axitinib (AG-013736), erlotinib, gefitinib, axitinib, temsirolimus and nilotinib (AMN107).

A PDGFR inhibitor may be preferably a PDGFRβ inhibitor.

Hence, the above described problem of the invention is also solved by a medicinal combination comprising (a) an inhibitor of the lncRNA as defined herein before (in particular a TYKRIL inhibitor), and (b) a PDGFR inhibitor, as defined above.

Such a combination is preferably used in the treatment of a disease such as described herein above.

Another embodiment of the invention then pertains to a PDGFR inhibitor for use in the treatment of a disease, wherein the treatment involves the simultaneous or sequential administration of an lncRNA inhibitor according to any of the preceding claims.

The above mentioned uses of compounds are further applied in methods for treating a subject in need of such a treatment, wherein the method comprises the administration of the inhibitors or agonists to subject in a therapeutically effective amount.

The agonists or inhibitors as described herein above are useful in the treatment of the various diseases mentioned above. Therefore the present invention provides the use of the compounds of the invention in a curative or prophylactic medical treatment involving the administration of a therapeutically effective amount of the compound to a subject in need of such a treatment.

The agonists or antagonists described may be used alone or in combination with other methods for treating of the various diseases associated with angiogenesis. For example if a subject has been diagnosed with cancer, the one or more agents described above may be combined with administration of a therapeutically effective amount of a compound that is therapeutically active for the treatment of this cancer, for example a chemotherapeutic agent.

The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing or treating a target condition, alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

The term “in combination” or “in combination with,” as used herein, means in the course of treating the same disease in the same patient using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

The term “subject” as used herein means a human or other mammal. In some embodiments, the subject may be a patient suffering or in danger of suffering from a disease as disclosed herein.

Hence, furthermore provided are pharmaceutical compositions, comprising an inhibitor of an lncRNA as disclosed, a combination as disclosed or a PDGFR inhibitor as disclosed, optionally together with a pharmaceutical acceptable carrier and/or excipient.

As used herein the language “pharmaceutically acceptable carrier” is intended to include any and all solvents, solubilizers, fillers, stabilizers, binders, absorbents, bases, buffering agents, lubricants, controlled release vehicles, nanoparticles, liposomes, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the inject-able compositions can be brought about by including in the composition an agent which delays absorption, for example, aluminum monostearate and gelatin.

Sterile injectable solutions can be prepared by incorporating the active compound (e.g., a neuregulin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) or nanoparticles, including those prepared with poly(dl-lactide-co-glycolide), can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser together with instructions for administration.

An inhibitor of an lncRNA in accordance of the invention may in a preferred embodiment be formulated to be contained within, or, adapted to release by a surgical or medical device or implant. In certain aspects, an implant may be coated or otherwise treated with the compounds of the invention. For example, hydrogels, or other polymers, such as biocompatible and/or biodegradable polymers, may be used to coat an implant with the compounds of the present invention, or compositions containing them (i.e., the composition or pharmaceutical composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing. The invention pertains to the use of the modulators of the lncRNA of the invention in the manufacture of surgical or medical devices as well as to the so modified surgical or medical device or implants as such. The devices and implants of the invention are useful for a controlled and spatially restricted administration of the modulators of lncRNA of the invention at the site of action, which is the targeted tissue or organ, for example a blood vessel or the heart.

The present invention is in another aspect provides an in vitro method for screening a modulator of the expression and/or function of a lncRNA selected from TYKRIL (also known as AP001046.5), MIR210HG, RP11-367F23.1, H19, RP11-44N21.1, AC006273.7, RP11-120D5.1, RP11-443B7.1, AC005082.12, RP11-65J21.3, or AC008746.12, the method comprising,
(a) Providing a sample of pericytes,
(b) Optionally, Induce hypoxia in the sample of pericytes,
(c) Contact the sample of pericytes with a candidate compound,
(d) Determine at least one of the following in the sample of pericytes:
   (i) The expression level of the lncRNA,
   (ii) The expression level of PDGFR,
   (iii) recruitment of the pericytes towards endothelial cells,
   (iv) proliferation of pericytes,
   (v) activity or expression of p53
   (vi) interaction of p53 with the histone acetyltransferase p300

wherein a significant change in any of (i) to (iv) compared to a control indicates that the candidate compound is a modulator of the lncRNA expression and/or function.

This method is preferably used to identify an inhibitor to be used in context of the aforedescribed embodiments.

Preferably a reduced expression in (i) and/or (ii) compared to a control, and/or an impaired recruitment in (iii) compared to a control, and/or a reduced proliferation in (iv), and/or altered expression or activity of p53 in (v), and/or altered interaction of p53 with its co-activator p300 in (vi) indicates that the candidate compound is an inhibitor of lncRNA expression and/or function.

Most preferred is the above screening method, wherein in step (d) at least (i) is determined.

For skilled artisan it is apparent that in the screening method step (b) and step (c) may be performed in reverse order or simultaneously.

In context of the invention expression levels of lncRNA of the invention are preferably determined via quantitative PCR analysis which is well known to the person of skill in the art. In order to detect pericyte recruitment it is preferred to perform a matrigel assay, for example, as described in the example sections.

The method is in another embodiment, for identifying an inhibitor of an lncRNA selected from selected from TYKRIL (also known as AP001046.5), MIR210HG, RP11-367F23.1, H19, RP11-44N21.1, AC006273.7, RP11-120D5.1, RP11-443B7.1, AC005082.12, RP11-65J21.3, or AC008746.12.

As mentioned herein above, the invention also provides agonists of the lncRNA of the invention. Such agonists may in preferred embodiments be selected from lncRNA expression constructs.

Aspects of the present invention relate to various vehicles comprising the nucleic acid molecules, preferably the antisense or lncRNA molecules, of the present invention. By vehicle is understood an agent with which genetic material can be transferred. Herein such vehicles are exemplified as nucleic acid constructs, vectors, and delivery vehicles such as viruses and cells.

By nucleic acid construct or expression construct is understood a genetically engineered nucleic acid. The nucleic acid construct may be a non-replicating and linear nucleic acid, a circular expression vector, an autonomously replicating plasmid or viral expression vector. A nucleic acid construct may comprise several elements such as, but not limited to genes or fragments of same, promoters, enhancers, terminators, poly-A tails (usually not necessary for lncRNA), linkers, markers and host homologous sequences for integration. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Furthermore, the present invention provides modified nucleic acids, in particular chemically modified RNA (modRNA) that can be used directly for the delivery of an lncRNA sequence of the invention (Zangi L. et al, “Modified mRNA directs the fate of heart progenitor cells and induces vascular regeneration after myocardial infarction” 2013, Nat Biotechnol). Such modified RNA may be produced by use of 3′-O-Me-m7G(5′)ppp(5′)G cap analogs and is described in Warren L, Manos P D, Ahfeldt T, et al. Highly efficient reprogramming to pluripotency and directed differentiation of human cells with synthetic modified mRNA. Cell Stem Cell. 2011; 7:618-630.

Several nucleic acid molecules may be encoded within the same construct and may be linked by an operative linker. By the term operative linker is understood to refer to a sequence of nucleotides that connects two parts of a nucleic acid construct in a manner securing the expression of the encoded nucleic acids via the construct.

The term promoter will be used here to refer to a group of transcriptional control modules that are clustered around the initiation site for RNA polymerase. The best known example of this is the TATA box, but in some promoters lacking a TATA box, such as the promoter for the mammalian terminal deoxynucleotidyl transferase gene and the promoter for the SV 40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription. Any promoter that can direct transcription initiation of the sequences encoded by the nucleic acid construct may be used in the invention.

An aspect of the present invention comprises the nucleic acid construct wherein the sequence of at least one nucleic acid molecule is preceded by a promoter enabling expression of at least one nucleic acid molecule.

It is a further aspect that the promoter is selected from the group of constitutive promoters, inducible promoters, organism specific promoters, tissue specific promoters and cell type specific promoters. Examples of promoters include, but are not limited to: constitutive promoters such as: simian virus 40 (SV40) early promoter, a mouse mammary tumour virus promoter, a human immunodeficiency virus long terminal repeat promoter, a Moloney virus promoter, an avian leukaemia virus promoter, an Epstein-Barr virus immediate early promoter, a Rous sarcoma virus (RSV) promoter, a human actin promoter, a human myosin promoter, a human haemoglobin promoter, cytomegalovirus (CMV) promoter and a human muscle creatine promoter, inducible promoters such as: a metallothionine promoter, a glucocorticoid promoter, a progesterone promoter, and a tetracycline promoter (tet-on or tet-off), tissue specific promoters such as: HER-2 promoter and PSA associated promoter.

An aspect of the present invention comprises the nucleic acid construct as described in any of the above, comprised within a delivery vehicle referred to as vector. A delivery vehicle is an entity whereby a nucleotide sequence can be transported from at least one media to another. Delivery vehicles are generally used for expression of the sequences encoded within the nucleic acid construct and/or for the intracellular delivery of the construct. It is within the scope of the present invention that the delivery vehicle is a vehicle selected from the group of: RNA based vehicles, DNA based vehicles/vectors, lipid based vehicles, virally based vehicles and cell based vehicles. Examples of such delivery vehicles include, but are not limited to: biodegradable polymer microspheres, lipid based formulations such as liposome carriers, coating the construct onto colloidal gold particles, lipopolysaccharides, polypeptides, polysaccharides, pegylation of viral vehicles.

A preferred embodiment of the present invention comprises a virus as a delivery vehicle, where the virus is selected from the non-exhaustive group of: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

Provided are also uses of the aforementioned agonists or inhibitors of the herein disclosed lncRNAs for modulating the function of pericytes in vitro. The modulation may affect positively or negatively, depending on whether an agonist or inhibitor is used, the proliferation, PDGFR expression or endothelial cell recruitment of the pericyte.

The lncRNA of the present were identified to be up-regulated upon hypoxia, and therefore are indicative for cardiovascular ischemia and tumor hypoxia. Hence, the lncRNA of the invention are further useful as diagnostic markers. Therefore the invention in another aspect provides a method for stratification, monitoring or diagnosing cardiovascular ischemia or tumor hypoxia in patient, the method comprising the steps of (a) providing a sample of the patient, (b) determining the level of at least one lncRNA selected from TYKRIL (also known as AP001046.5), MIR210HG, RP11-367F23.1, H19, RP11-44N21.1, AC006273.7, RP11-120D5.1, RP11-443B7.1, AC005082.12, RP11-65J21.3, or AC008746.12, wherein an increased level of the lncRNA compared to a healthy control indicates cardiovascular ischemia or tumor hypoxia in the patient. In particular the cardiovascular ischemia or tumor hypoxia is indicative for the presence of a cardiovascular disease or respectively tumorous disease as described herein elsewhere.

The step of “providing a sample” from the patient shall be understood to exclude any invasive procedures directly performed at the patient. Therefore the diagnostic method of the invention is preferably a non-invasive method, such as an ex vivo or in vitro diagnostic method.

The step of determining the level of the lncRNA preferably comprises the use of at least one primer or probe identical or complementary to the sequence of an lncRNA of the invention. The person of skill using the knowledge of the present invention may without harnessing inventive activity design primers or probes in order to detect the expression level of the at least one lncRNA for the diagnostic purposes disclosed.

The term “patient” in context of the present invention in all of its embodiments and aspects is a mammal, preferably a human.

The term “sample” is a tissue sample, for example heart/lung/brain/kidney/liver/spleen tissue sample, or a liquid sample, preferably a blood sample such as a whole blood sample, serum sample, or plasma sample, or a tumor sample.

The term “healthy control” in context of the diagnostics of the invention corresponds to (i) the level of the one or more lncRNA in a sample from a subject not suffering from, or not being at risk of developing, the cardiovascular ischemia or tumor hypoxia, or (ii) the level of the one or more lncRNA in a sample from the same subject at a different time point, for example before or after conducting a medical treatment. The latter control is specifically useful for monitoring purposes.

For patient stratification in context of the invention the lncRNA level may be detected according to the diagnostic method in the sample of a patient. In case the lncRNA is up-regulated in the sample compared to the control, the patient's tumor qualifies for a treatment using an inhibitor of the lncRNA in accordance with the herein disclosed aspects relating to lncRNA inhibitors and therapeutic uses.

Furthermore, the diagnostic method may be applied in order to monitor treatment success in a patient. For this purpose the diagnostic method of the invention is repeated at regular timepoints in order to observe whether the applied treatment successfully reduced cardiovascular ischemia or tumor hypoxia the patient.

In order to determine the level of the lncRNA in the sample of the patient the person of skill in the art may use any methods applicable for directly or indirectly quantifying RNA molecules. This includes techniques such as ELISA, fluorescence in situ hybridization (FISH), flow cytometry, flow cytometry-FISH, antibodies against the lncRNA, in situ hybridization and quantitative PCR techniques.

A preferred diagnostic lncRNA of the invention is TYKRIL. Thus, preferred primers and probes of the invention are those sequences as disclosed in the example section for the detection of TYKRIL expression. However, the present invention shall not be understood to be limited to those specifically preferred embodiments.

The present invention will now be further described in the following examples with reference to the accompanying figures and sequences, nevertheless, without being limited thereto. For the purposes of the present invention, all references as cited herein are incorporated by reference in their entireties. In the Figures:

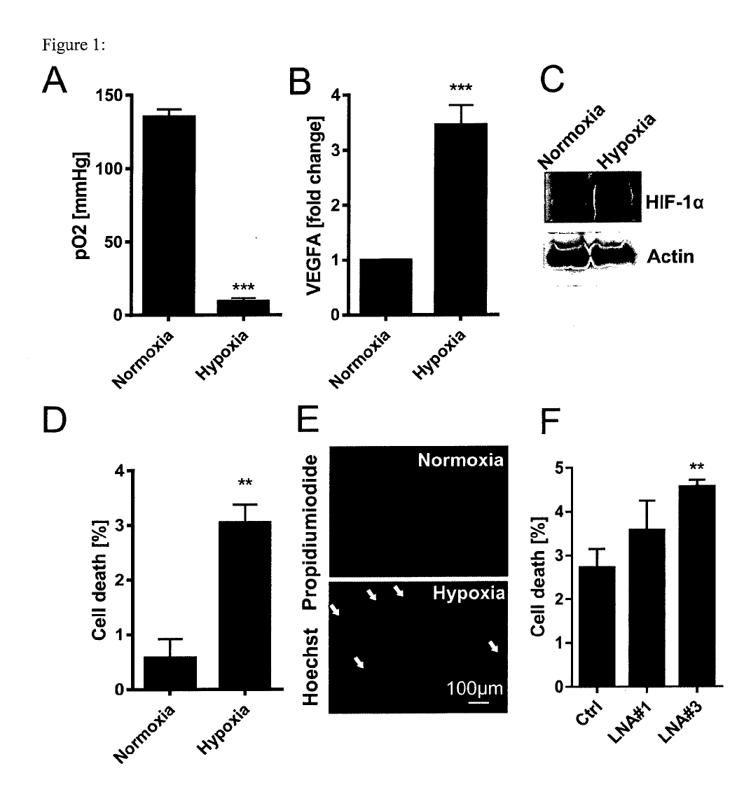

FIG. 1: A 24 h 1% O2 resulted in an efficient reduction of pO2 levels in PC culture as determined by pO2 measurements (n>3). B In order to control the efficacy of hypoxic cell responses, VEGFA levels were determined which were significantly increased upon hypoxic treatment (n=7). C Upon hypoxia HIF-1a was upregulated in PC. D 24 h hypoxia resulted in a sparse increase of cell death as determined by PI and Hoechst counterstains (n=3-4) E. TYKRIL knockdown resulted in a sparse increase of cell death as determined by flow cytometry for PI positive PC (n=4) F. (*** P<0.001; ** P<0.01; * P<0.05)

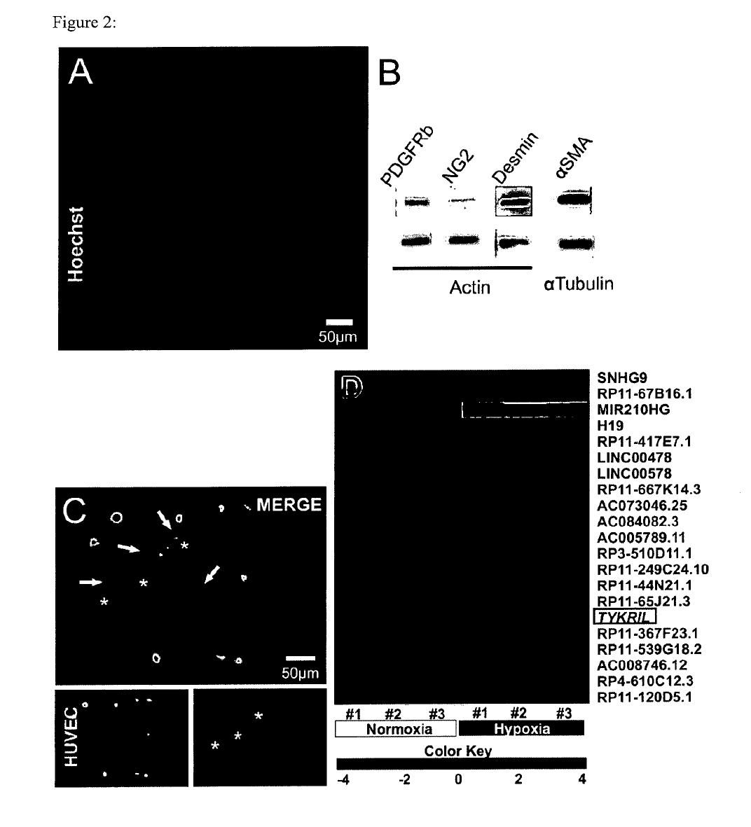

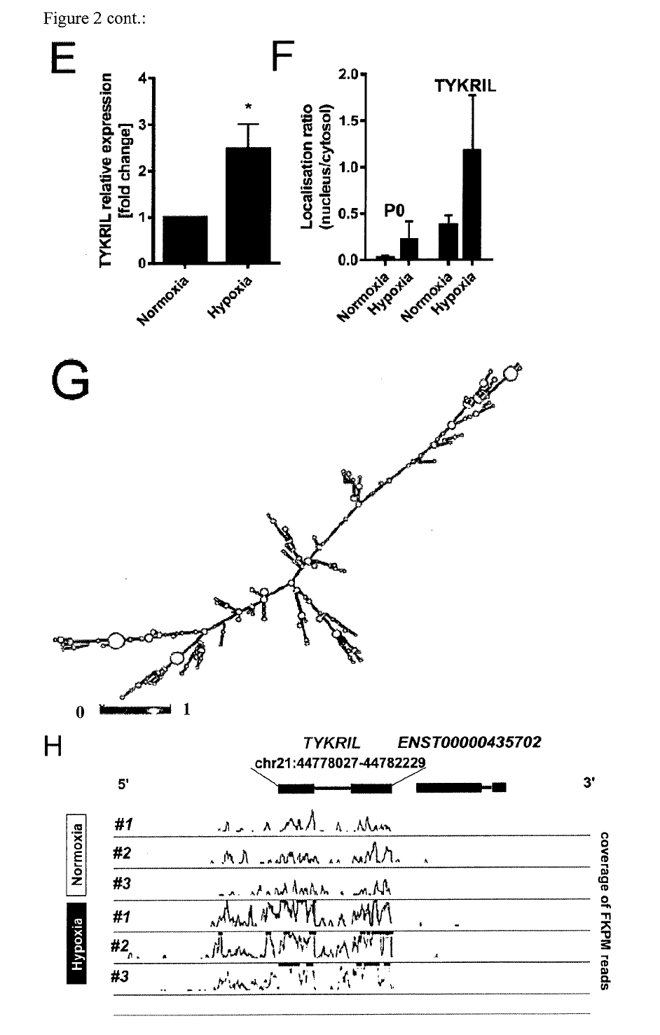

FIG. 2: Characterization of PC and TYKRIL: A Immunostainings show that PC used in the present study robustly express PDGFRβ (red) on protein level (representative confocal z-stack, maximum projection from at least n=3 experiments). B Immunoblots depict robust expression of the PC markers PDGFRβ, NG2, Desmin and αSMA in PC lysates. C Coculture experiments between HUVEC (green) and PC (cells marked with asterisks in red) indicate intercellular dye transfer between both cell types indicated by yellow cells marked by arrows. D A heatmap depicting the top regulated lncRNAs upon Hypoxia including TYKRIL (n=3 experiments per condition, P<0.05). E Upregulation of TYKRIL was confirmed by qRT-PCR. F TYKRIL is located in both nuclear and cytosolic cellular fractions under hypoxia and normoxia (n=4, no significant differences). Panel G depicts the estimated secondary structure of TYKRIL (lnci-pedia.org). H Analyses of the RNA deep sequencing reads shows a high coverage of exons 1 and 2 of TYKRIL which is located on chr2l next to transcript ENST00000435702. (* P<0.05).

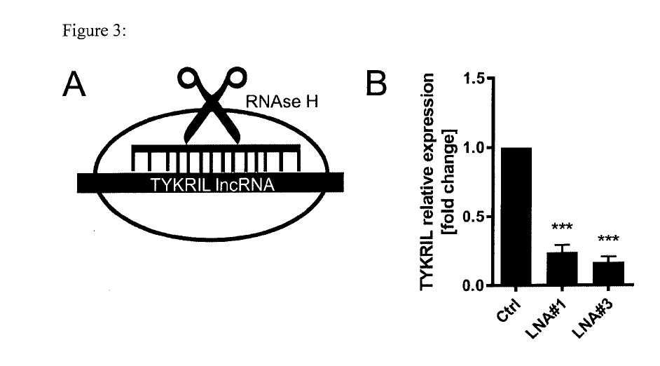

FIG. 3: TYKRIL silencing strategy: A LNA GapmeRs specifically binding to TYKRIL were designed. Upon binding TYKRIL is cleaved by RNAse H within the nucleus. B In order to minimize possible unspecific off-side effects by LNA GapmeRs, 2 distinct sequences were used to silence TYKRIL which effectively lowered TYKRIL expression levels in PC. (*** P<0.001)

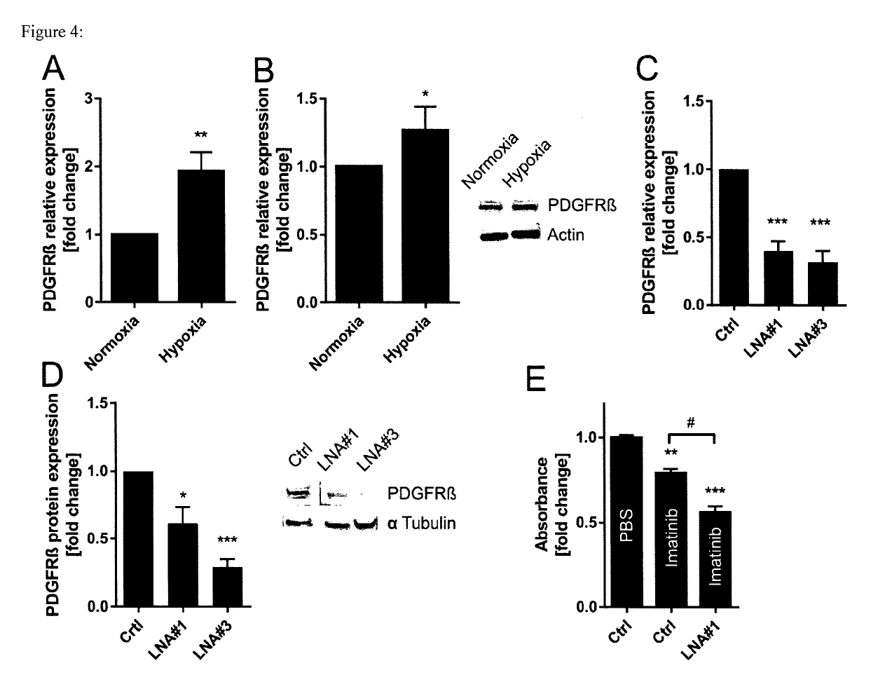

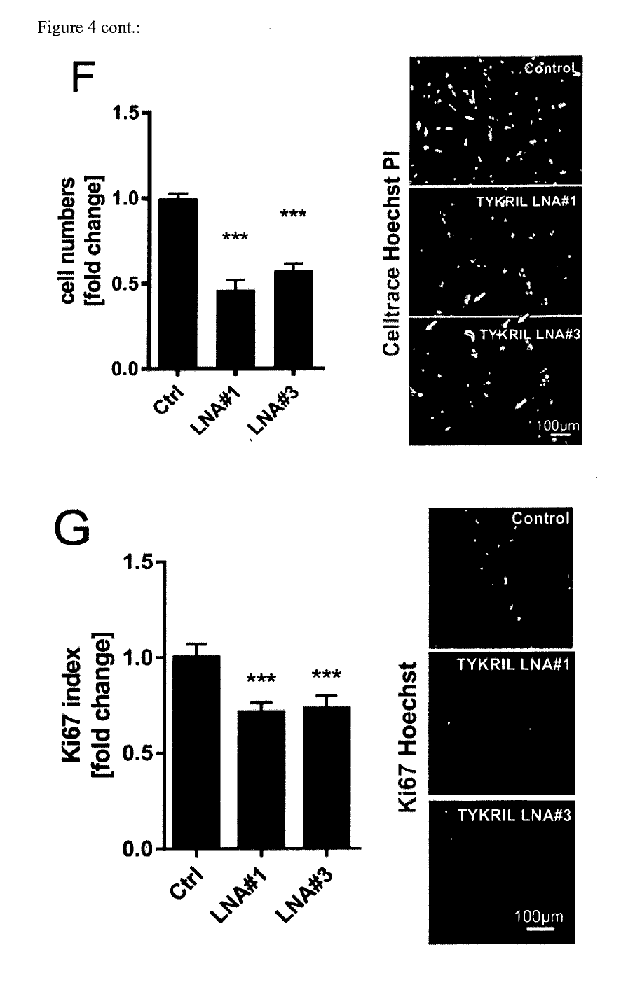

FIG. 4: Impact of hypoxia and TYKRIL on PDGFRβ and PC function: A Hypoxia resulted in an increase of PDGFRβ on mRNA and B protein level. C TYKRIL silencing significantly reduced PDGFRβ gene expression as well as D PDGFRβ protein expression. E Imatinib (1 μM, 24 h) significantly decreased PC viability compared with PC treated with the solvent PBS. Upon TYKRIL silencing, PC viability was significantly more reduced by imatinib treatment compared with PC transfected with scramble controls as determined by MTT assays (n=2-4). F Following TYKRIL silencing, PC cell numbers were significantly reduced 48 h after LNA transfection. Fluorescent images are representative images depicting PC (Calcein green CellTrace), cell nuclei (blue, Hoechst) and dead cells (arrowheads marking red nuclei, PI=propidiumiodide). G Ki67 stains in PC show a decrease in cell proliferation upon TYKRIL knockdown, images show representative images from n>3 per condition). (*** P<0.001; ** P<0.01; * P<0.05; ### P<0.001).

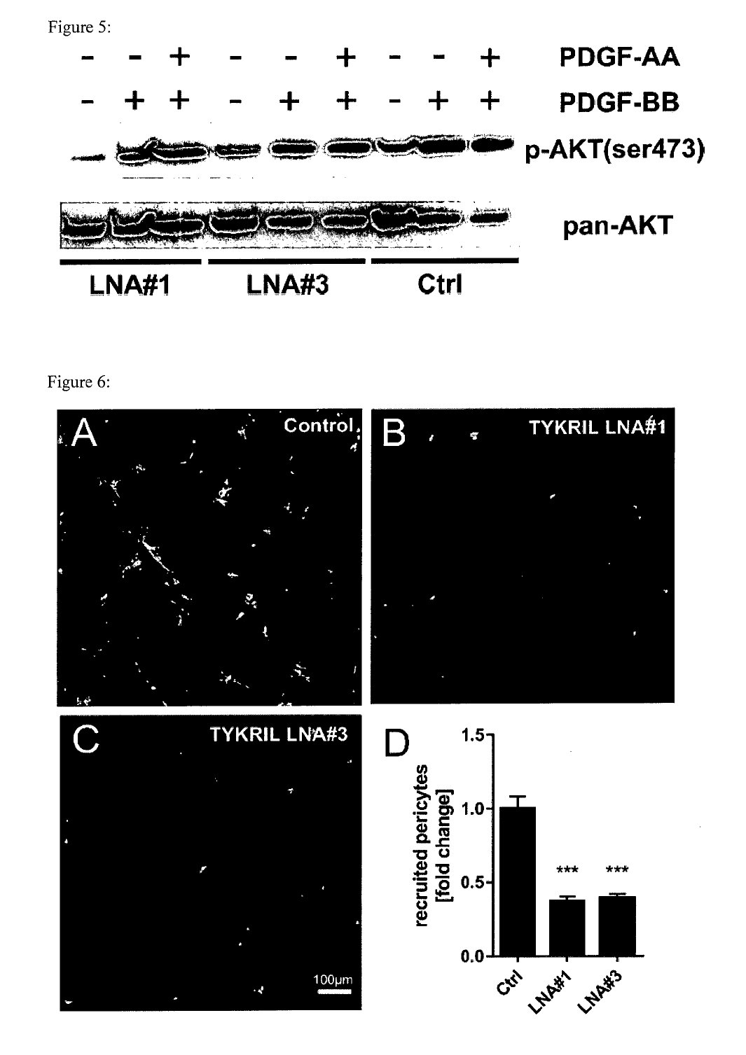

FIG. 5: TYKRIL silencing impairs PDGFRβ downstreaming phosphorylation of AKT: AKT, which is essential for cell proliferation and cell migration, is an established downstream signaling pathway of PDGFRβ stimulation with PDGF-BB. In PC that were treated with LNA GapmeRs against TYKRIL phosphorylation of AKT compared with solvent treated controls could be detected (upper lane). However, phosphorylation of AKT was markedly reduced compared with LNA GapmeR controls. This indicates an impaired PDGFRβ downstream signal transduction upon TYKRIL knockdown with regard to AKT. The same filter was stripped and a pan-AKT antibody was applied to visualize total AKT protein content.

FIG. 6: TYKRIL is essential for PC recruitment towards endothelial cells: A numerous GFP labelled PC treated with LNA GapmeR scramble control are covering HUVEC (red) tube formations in a perivascular manner. B, C Silencing of TYKRIL in PC results in a significant reduction of PC recruitment towards PC that is below 50% compared to controls D. (*** P<0.001).

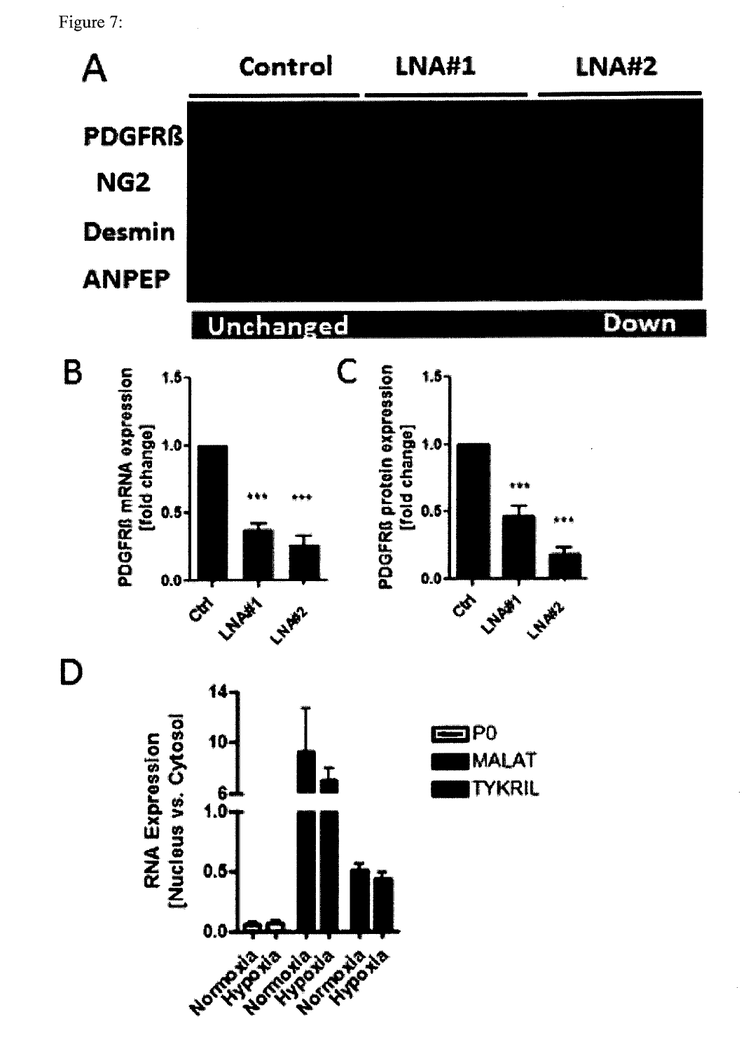

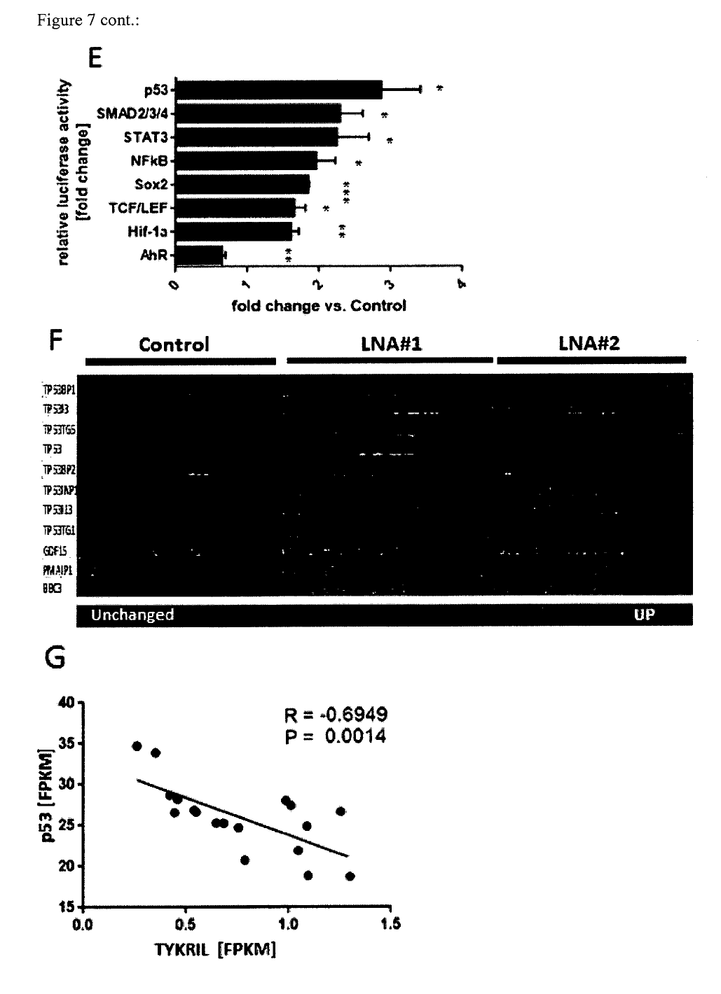

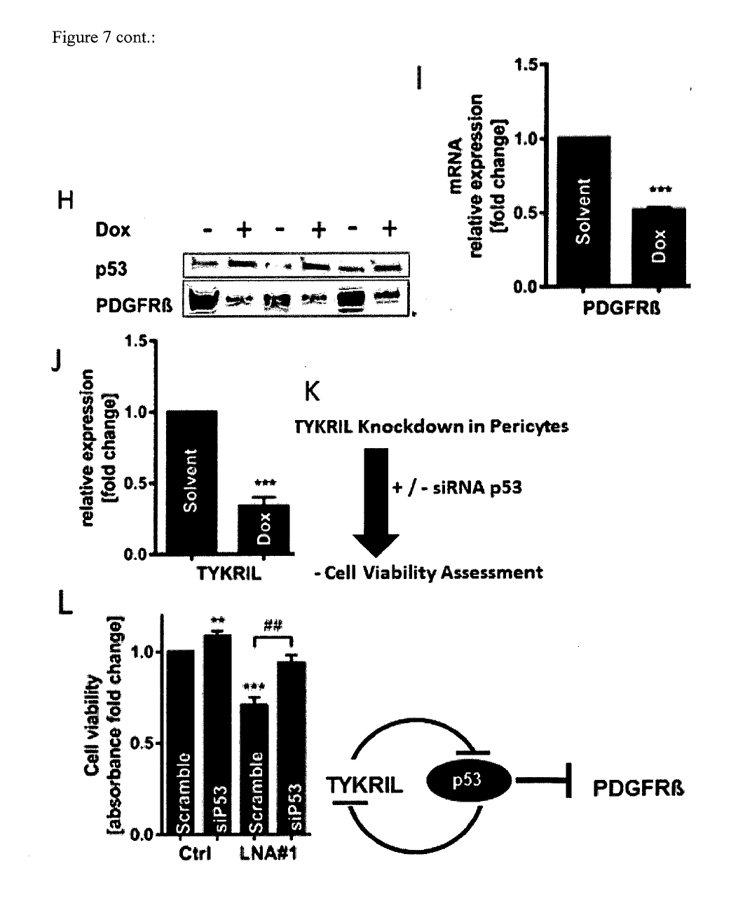

FIG. 7: RNA Seq upon TYKRIL knockdown with LNA GapmeRs LNA#1 and LNA#3 reveals de-differentiation of Pericytes (A). qPCR and immunoblotting confirm the loss of PDGFRβ upon TYKRIL knockdown (B, C). TYKRIL is localized in both, cytosol and nucleus of the cell (D) whilst transcription factor profiling demonstrates a prominent upregulation of p53 activity (E) which is confirmed by RNA seq which shows upregulation of p53 and its downstream target genes (F). Importantly, p53 expression negatively correlates with TYKRIL expression (G). p53 gain of function by doxorubicin treatment (Dox, H) results in a downregulation of PDGFRβ on protein (H), RNA level (I) and a loss of TYKRIL (J). Co-silencing p53 (K, L), rescues loss of cell viability upon TYKRIL knockdown indicating a regulatory feedback loop between TYKRIL and p53.

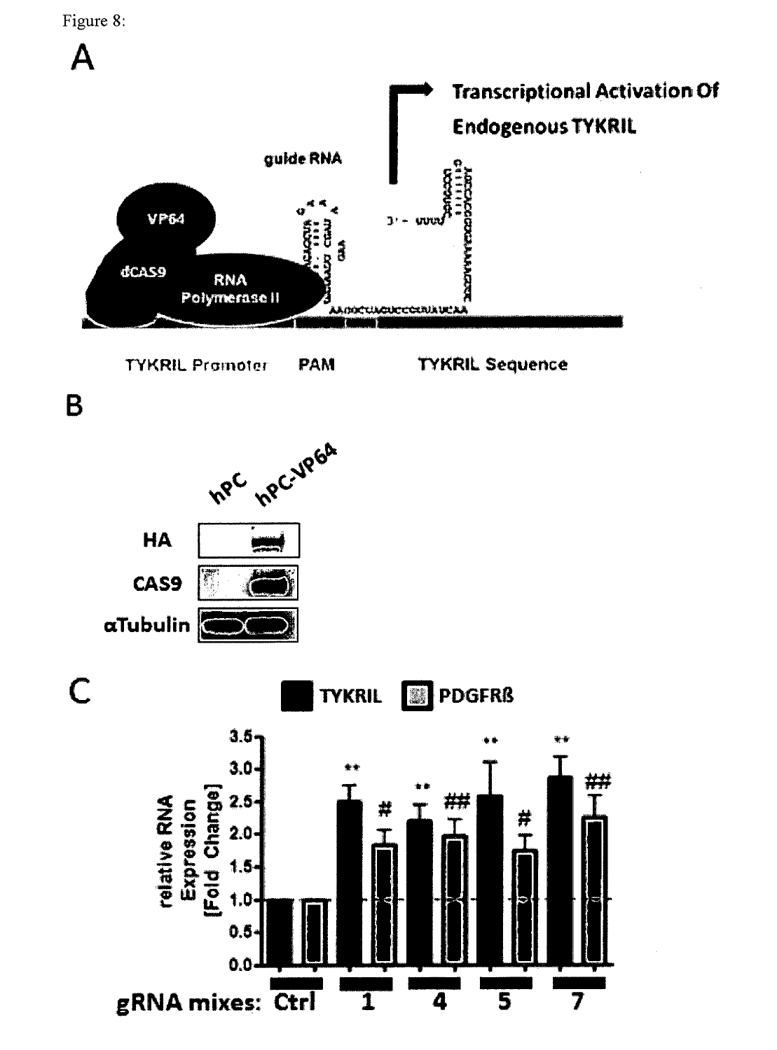

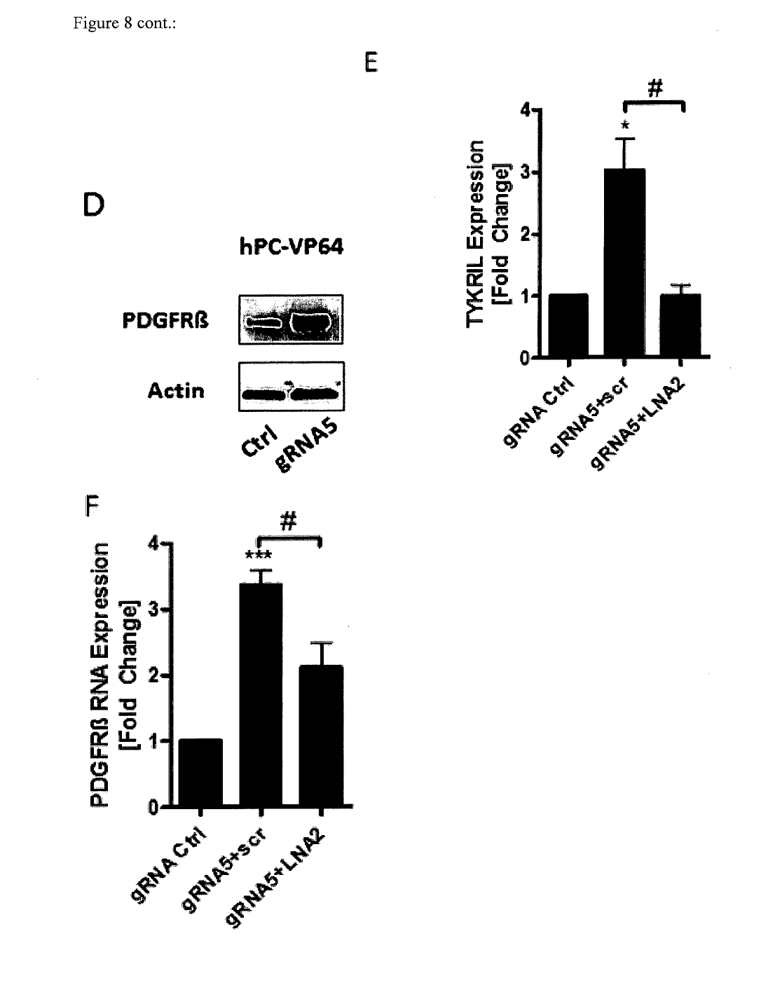

FIG. 8: Expression of RNA guides directed against the TYKRIL promoter region (A) in human pericytes that express HA-tagged inactive CAS9 carrying the transcriptional activator VP64 (hPC-VP64, B) result in a significant upregulation of TYKRIL and PDGFRβ (C) various guide RNA sequences were tested to minimize off-target effects. Transfection of gRNAs result in an upregulation of PDGFRβ on protein level (D). Co-transfection of gRNAs with LNA GapmeRs confirm the specificity of GapmeRs and gRNAs since co-transfection blocks gRNA mediated overexpression of TYKRIL (E) and partly PDGFRβ (F).

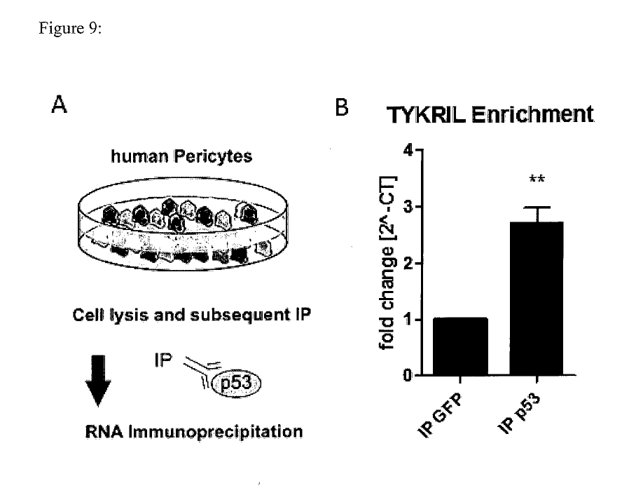

FIG. 9: Following UV crosslinking, cell lysis and IP for p53 (Control: anti-GFP IP, A), RNA immunoprecipitation was performed. TYKRIL was significantly enriched in IP p53 samples compared to GFP control (B) demonstrating physical interaction between TYKRIL and p53.

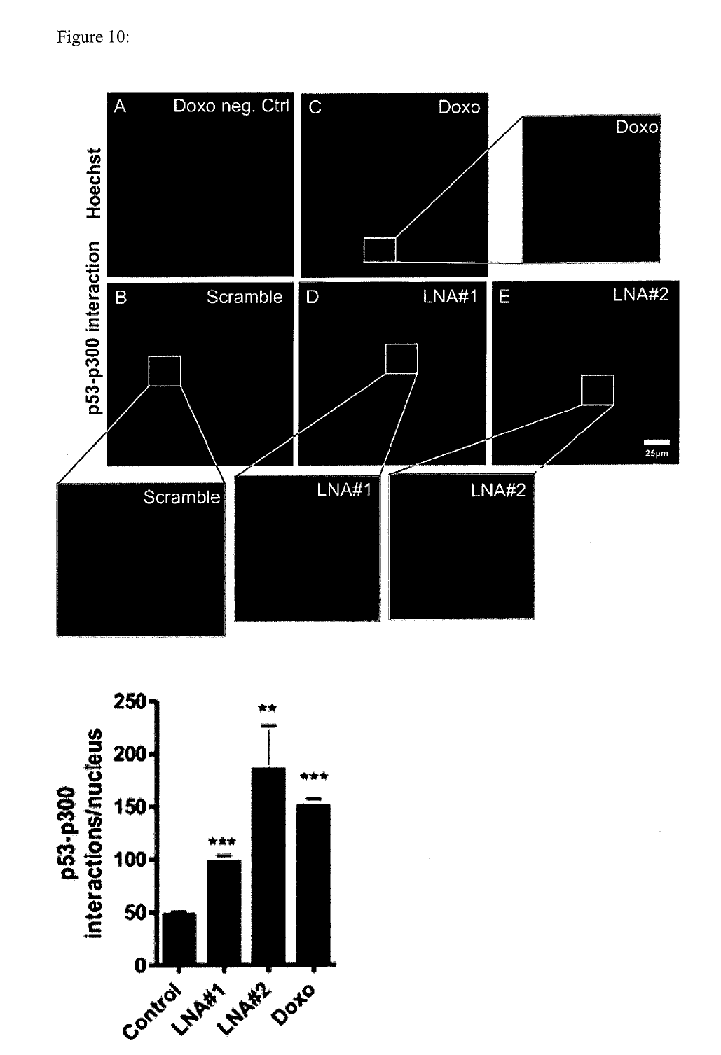

FIG. 10: Specific proximity ligation assays (negative control: A) demonstrate sparse p53-p300 interaction in scramble control compared with doxorubicin (positive control, C) treated human pericytes. Likewise, TYKRIL knockdown resulted in a significant increase of nuclear p53-p300 interaction.

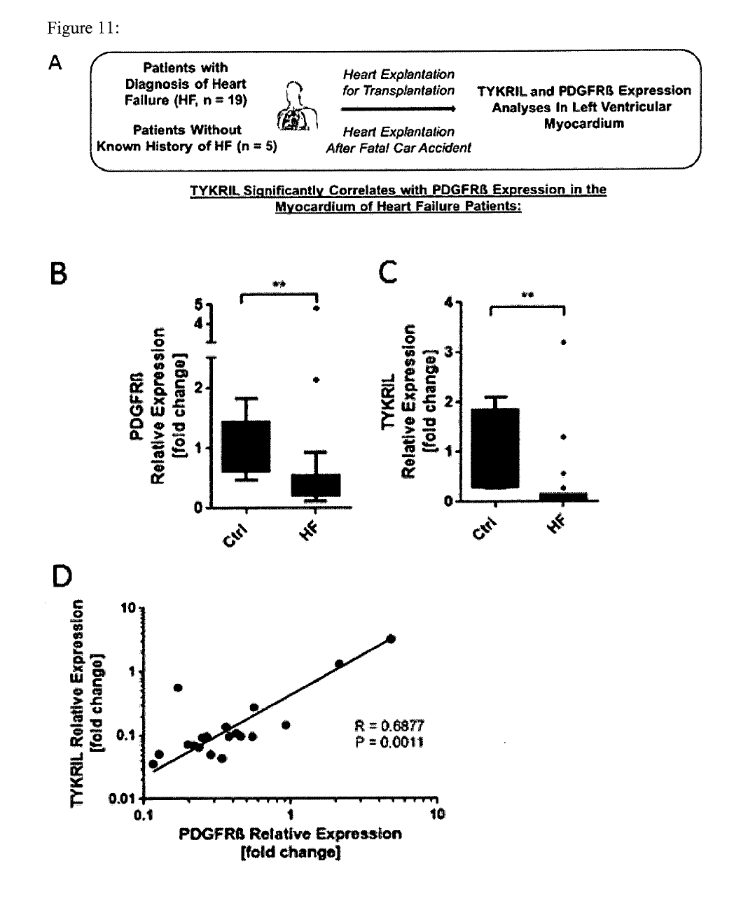

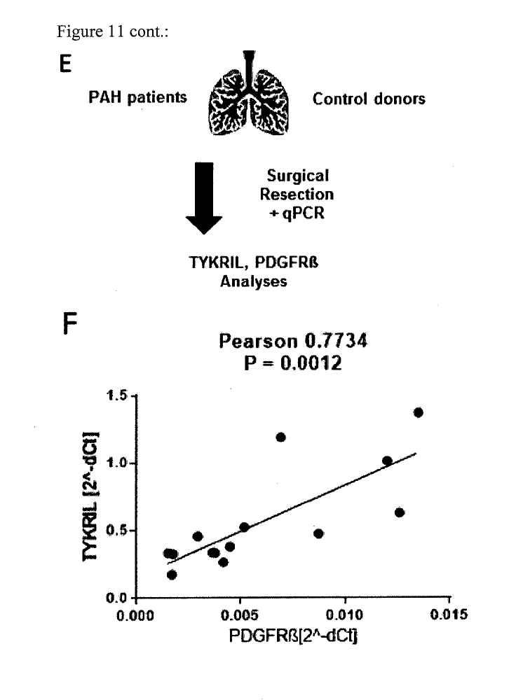

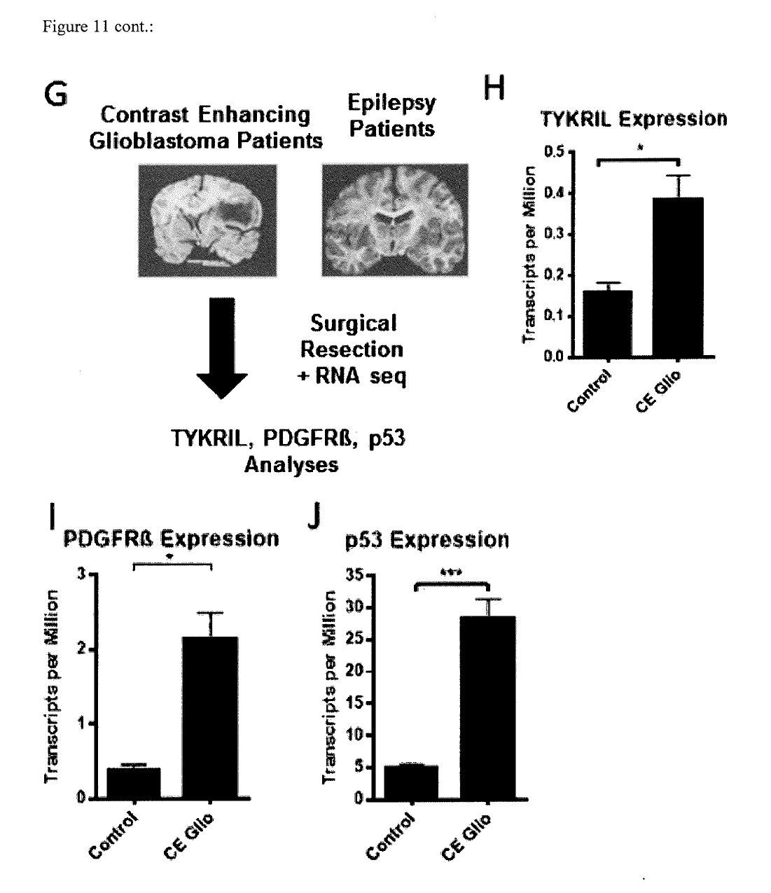

FIG. 11: TYKRIL was measured in patient cohorts from controls and patients diagnosed with heart failure (HF, A). PDGFRβ (B) as well as TYKRIL (C) were significantly less expressed in HF compared with control. TYKRIL and PDGFRβ expression significantly correlated with each other in HF (D). Likewise, TYKRIL-PDGFRβ analyses in PAH (n=7) and control lungs (n=7) reveals TYKRIL-PDGFRβ correlation in lung tissue (E, F) pointing to TYKRIL-PDGFRβ interdependence in the cardiopulmonary system in humans. RNA seq from glioblastoma samples (G) shows that TYKRIL and PDGFRβ are both significantly elevated in malignancy (H, I) whilst p53 is upregulated (J).

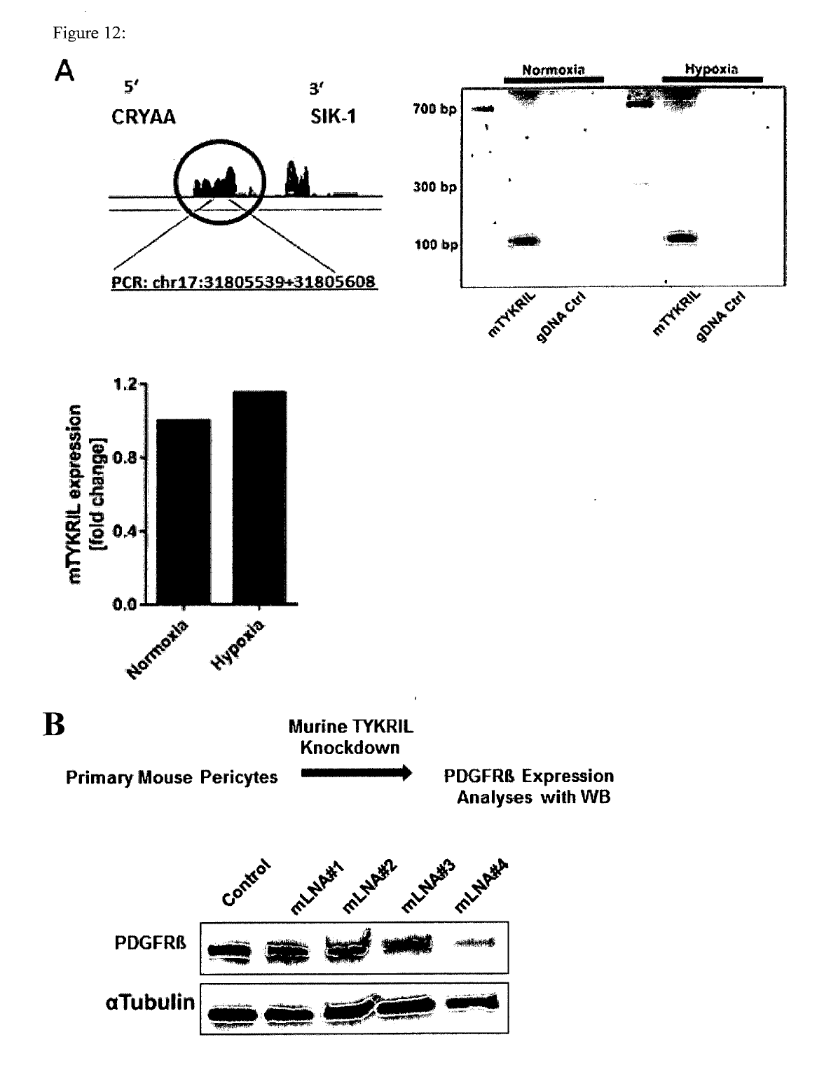

FIG. 12: PCR against locus conserved sequences with homologies to the human TYKRIL sequence unravel murine TYKRIL located+/−3 kp from genomic locus chr17:31805539-31805608 (GRCm38/mm10) (A). PCR in normoxic and hypoxic conditions demonstrate upregulation by hypoxia (A). Silencing the murine orthologue results in loss of PDGFRβ as demonstrated by 4 different LNA GapmeRs in immunoblotting (B).

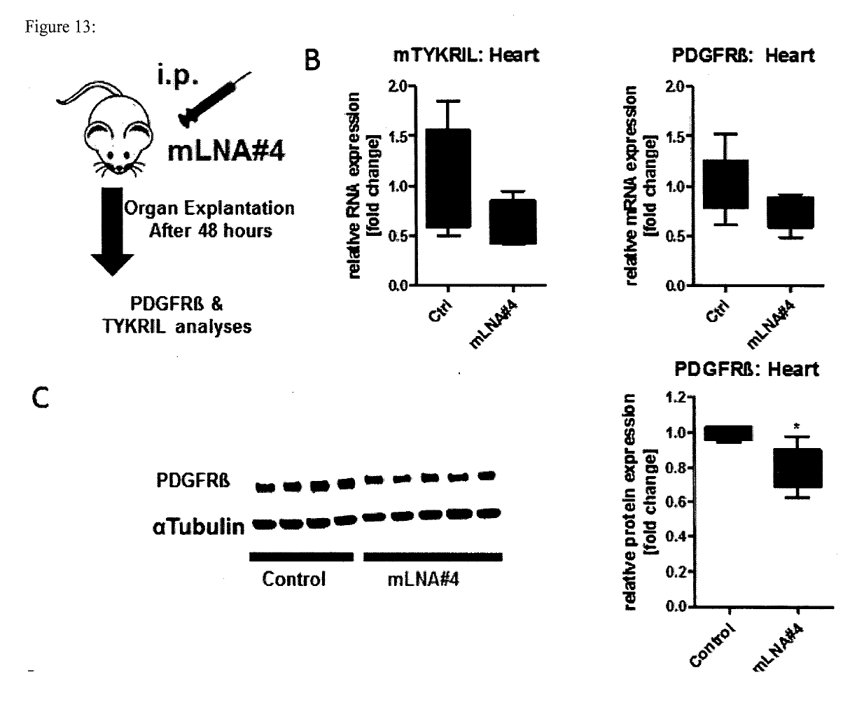

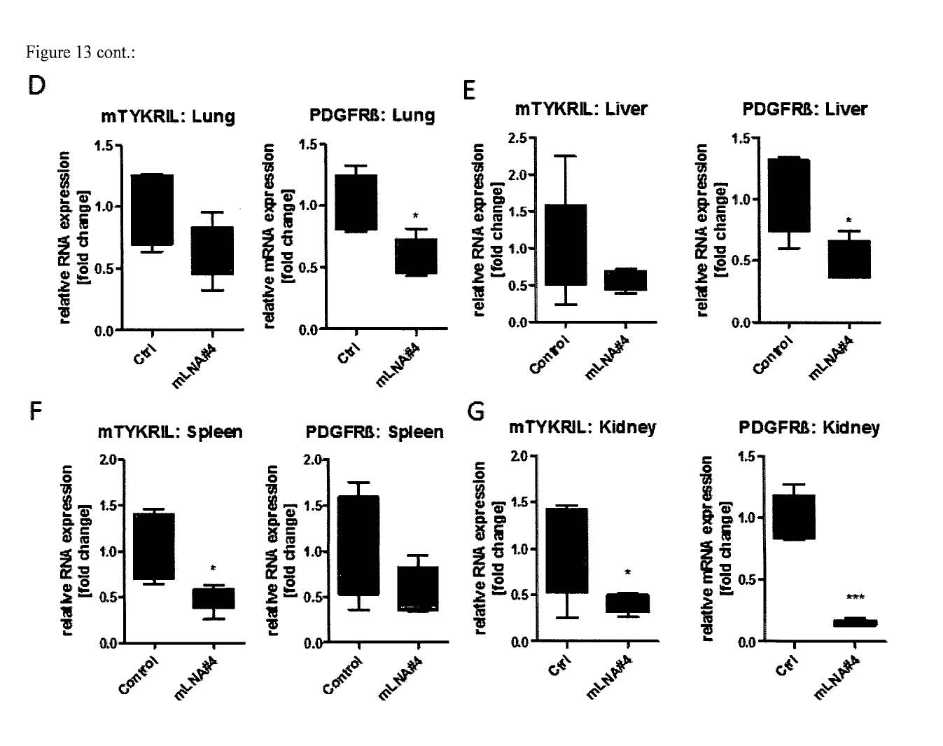

FIG. 13: Mice were injected mLNA#4 intraperitoneally and organs were harvested for PDGFRβ and TYKRIL analyses 48 h after injection (A). qPCR demonstrates downregulation of PDGFRβ and mTYKRIL in the HEART (B), PDGFRβ loss was also confirmed on protein level (B). mTYKRIL and PDGFRβ were also decreased in the lungs (D), liver (E), spleen (F) and kidneys (G). n=4-5 animals per group.

SEQ ID NO: 1 TYKRIL sequence (the TYKRIL genomic DNA sequence is provided. The TYKRIL RNA molecule contains the same sequence but as RNA, thus uracil instead of thymine)
SEQ ID NO: 2, 3, 4 TYKRIL LNA-GapmeR sequences, and control
SEQ ID NO: 5, 6 TYKRIL Primer
SEQ ID NO: 7, 8 murine TYKRIL Primer
SEQ ID NO: 9-12 murine TYKRIL LNA GapmeR sequences.

### EXAMPLES

Materials and Methods

Cell Culture

Human Pericytes (passages 2-9; from ScienCell, Carlsbad, Calif., USA) were cultured as recommended by the manufacturer. Cells were kept at 5% CO2, 20% O2, 37° C. and humidified atmosphere. hPC medium consisted of DMEM Glutamax (Gibco, Life Technologies, Carlsbad, Calif., USA) supplemented with Penicillin/Streptomycin (Roche Diagnostics) and 10% fetal calf serum. HUVEC were cultured as described previously in detail 22.

Induction of Hypoxia

Hypoxia was induced using a hypoxic incubator (Labotect, Gottingen, Germany). Cell culture medium was pre-equilibrated ahead of use overnight at 1% O2, 5% CO2 in a humidified atmosphere. Normoxic cell culture medium was carefully witched to hypoxic medium and hypoxic pO2 levels were verified by measuring pO2 levels with a hypoxia sensing probe from Oxford Optronix (Oxford, UK) as described in detail before 32. PCs were kept in hypoxic atmosphere for 24 hours. Experimental manipulations were carried after 24 h.

Transfection

Cells were grown to 60-80% confluency and were transfected for 4 hours in Optimem Medium using Lipfectamine (both from Life technologies, Carlsbad, Calif., USA) with 50 nmol/l LNA GapmeR (Exiqon, Vedbaek, Denmarkt) according to the manufacturer's instructions. Controls were transfected with scrambled LNA GapmeR control. Four hours after LNA Gapmer Transfection, Optimem Medium was exchanged with normal cell culture medium. All experimental manipulations were carried out 48 hours after transfection.

Matrigel Coculture Assays

Human pericytes expressing a green fluorescent protein were created by viral transduction with a lentivirus according to standard transduction procedures. A detailed transduction protocol is available upon request. Following transduction, PCs were treated with LNA GapmeRs or scramble Control. Endothelial cells are known to specifically take up acetylated LDL33. In order to label specifically endothelial cells, HUVEC were stained with acetylated Dil-LDL overnight (10 μg/ml, Cellsystems) ahead of coculture procedures. For Matrigel assays, extracellular matrix gel was thawed on ice. Subsequently 150 μl of cold gel was carefully transferred into a pre-cooled 24 Well Plate (Corning) using a pipette. 100.000 HUVEC were applied to the gel and incubated for 3 hours at 37° C., 5% CO2. Afterwards 10.000 GFP-expressing PC were added. After another 3 hours of incubation, PC medium was carefully removed and another part of cold gel was transferred with care into the well. After 30 minutes, another 500 μl cell culture medium was applied. After incubation overnight cells within the gels were fixed for 10 minutes in 4% PFA (Roti-Histofix, Carl Roth) and 3 randomly chosen field per view per probe were acquired using confocal imaging. Recruited pericytes were defined as GFP positive cells adhering to the HUVEC endothelial membrane. Recruited PC per field per view were counted using the Fiji Cell counter tool. Relative changes in PC recruitment related to Ctrl are presented.

Intercellular Dye Transfer Assay

HUVEC were seeded into a six well plate. At 60-80% confluency, HUVEC were stained with CellTrace calcein green AM (Lifetechnologies) according to the manufacturer's instructions. PC were grown to confluency in a culture dish and labelled with CellTrace calcein red AM (10 μmol/l, 30 minutes, Lifetechnologies). Subsequently, PC were washed, trypsinized and transferred into the HUVEC grown culture 6 W plate (30.000 PC/well). After 7 hours of coculture live cell imaging was performed.

RNA Deep Sequencing

RNA deep sequencing was performed by analyzing ribosomal depleted total RNA from human Pericytes. RNA was isolated using a RNeasy Mini Kit (Qiagen) according to the manufacturer's instructions including DNA digestion. Subsequently RNA was fragmented and primed for cDNA synthesis. Libraries were created using a TruSeq RNA Ribo-Zero Globin kit (from Illumina) as recommended by the manufacturer. A Illumina HiSeq 2000 flowcell was used for sequencing. lncRNA annotation was done based on the NONCODE database (noncode.org).

Quantitative Real-Time PCR (qRT-PCR) and RNA Isolation

Total RNA from PC was isolated using RNeasy Mini Kits (Qiagen, Hilden, Germany) as recommended by the manufacturer including a DNA digestion step. Nuclear and cytosolic fractions were prepared as documented elsewhere 34. 500-1000 ng RNA was reversely transcribed with random hexamer primers (ThermoScientific, Waltham, USA) by MulV reverse transcriptase (Lifetechnologies) in 40 μl reaction volume. Fast SYBR green or SYBR green (Applied Biosystems, Forster City, USA) and cDNA were used for qRT-PCR. A Viia7 or StepOnePlus from Applied Biosystems was used. CT values were normalized against ribosomal RPLP0. Relative gene expression levels were determined by the formula: 2-deltaCT; deltaCT=CTtarget−CTcontrol.

Flow Cytometry

Flow cytometry analyses were carried out according to standard procedures in a FACSCento II (BD Biosciences). A detailed protocol for the respective propidiumiodide staining procedures is available upon request.

Protein Isolation, SDS-Page and Western Blotting and Immunofluorescence

Standard immunofluorescent staining procedures were carried out as described before 35-37. In brief, cells were washed with PBS, fixed in ice cold acetone for about 5 minutes. After washing cells were blocked for 2 hours at room temperature with 5% donkey serum (Dianova, Hambur, Germany), 0.3% Triton X-100 in PBS. Primary antibodies were incubated overnight in PBS containing 2% BSA, 0.1% Triton X-100. Secondary antibodies and Hoechst (Lifetechnologies, 1/1000) were incubated in 2% BSA, 0.1% azide for another 2 hours. For SDS Page protein isolation cells were washed once with ice-cold PBS, snap frozen in liquid nitrogen and RIPA-buffer (Thermo Scientific, Rockford, USA) supplemented with protease inhibitor (Roche Diagnostics) was applied. Cells were then scraped off with an ice cold rubber policeman and incubated for 45 minutes on ice under agitation. Subsequently probes were centrifuged for 10 minutes at 5000 RPM at 4° C. The supernatant was transferred into ice cold vials and protein concentration was determined by performing a Bradford assay with RotiQuant (Carl Roth, Karlsruhe, Germany) according to the manufacturer's instructions. Protein samples were mixed with an equal volume of 2× Laemmli buffer (Sigma Aldrich). Gels (Mini-Protean TGX, BioRad) were loaded with 20-30 μg protein per lane. SDS page was performed for 1 hour at 100V in TBST (BioRad). Western Blotting was performed using a Pierce G2 Fast Blotter according to the manufacturer's instructions (ThermoScientific).

PDGF Stimulation

Pericytes were grown to 80% confluency and PDGF stimulation was performed as described before 2. Subsequently PC were starved for 1 h in serum-free PC culture medium. After starvation PC were treated with 100 ng/ml PDGF-AA (from Sigma Aldrich) or solvent control for 2 hours. Afterwards PC were stimulated with PDGF-BB (from Sigma, 30 ng/ml) for another 5 minutes. Finally protein content from PC were isolated and immunoblotting for phosphorylated AKT (ser473) was performed. The same filter was stripped and immunoblotted again with a pan-AKT antibody to visualize total AKT content in protein samples.

LNA Gapmer Transfection

LNA Gapmer transfection was performed as documented before in detail 22. In brief, cells were grown to 50-60% confluency. Subsequently transfection was performed with Lipofectamine (from Lifetechnologies) according to the manufacturer's instructions. GapmeRs or Control sequences were used at a concentration of 50 nmol/l. PC were briefly washed with OptiMEM medium (from Gibco), then PC were incubated with the transfection mixture for 4 hours in OptiMEM medium. Finally the OptiMEM medium containing the transfection agents was removed and PC were incubated for another 48 hours in humidified atmosphere, 5% CO2, 37° C. in PC culture medium. The GapmeR sequences were as follows:

LNA #1:
5′-3′: AGAGGTGATTAAGGT
LNA #3.
5′-3′: AGTGAAGGACAGAGGC
Control:
5′-3′: AACACGTCTATACGC

Antibodies

Primary antibodies: anti-PDGFRβ (Neuromics, GT15065, wb: 1/2000; IF: 1/200), anti-α SMA (Abcam ab7817, wb 1/200; IF: 1/100), anti-Desmin (Abcam ab32362, wb: 1/300), anti-NG2 (Millipore AB 5320, wb: 1/1000), anti-Ki67 (Abcam ab15580, IF:1/200), anti-tubulin (ab6160, wb: 1/5000), anti-vWF (Abcam ab11713, IF: 1/200), anti-HIF1α (BD Transduction 610958, wb: 1/1000), anti-pan-AKT (Cell Signaling 9272, 1:1000); anti-phospho-AKT Ser473 (Cell Signaling 9271). Anti-p53 (Abcam ab179477 for proximity ligation assay), anti-p300 (ActivMotif #61401), Anti-p53 (Thermo Scientific, MA5-12557, 1:100 for immunoblotting), Anti-HA (Cell signaling, #2367s 1:1000), Anti-Cas9 (Cell signaling, #14697S 1:1000).

Secondary antibodies: anti-goat cy3 (Dianova 705-165-147, 1/200); anti-rabbit 647 (Dianova 647711-605-152, 1/200); anti-rabbit cy2 (Abcam ab150073, 1); anti-rabbit HRP (Abcam ab16284); anti-rat HRP (Abcam ab102265); anti-mouse HRP (ab97030); anti-goat HRP (Abcam ab97110):

Confocal Microscopy and Image Analyses

A Leica SP5 confocal setup (Leica Microsystems) was used for image analyses. Z-stacks were acquired at 2 μm step size or smaller. Excitation wavelengths were: 405 nm, 488 nm, 552 nm or 638 nm. Images were further analyzed and processed using Fiji is just ImageJ for windows. In order to analyze Ki67 or PI positive cells automated Fiji particle analyses were used. Ki67 or PI counts were related to Hoechst positive cell counts to determine the percentage of cells in G1, S, G2 and mitosis or dead cells respectively. A detailed step by step protocol of the automated cell count procedure is available upon request.

Statistical Analyses

Results are documented with mean+/−standard error of the mean (SEM). All experiments were carried out at least for 3 times per experiment and condition. Data were analyzed using GraphPad Prism 6 for windows (Graphpad, San Diego, Calif., USA) and microsoft excel. The null hypothesis was rejected at α<0.05. Datasets were checked for normalization using a Pearson and D'Agostino omnibus method. In case of gaussian distribution, datasets were analyzed using an unpaired two sided student's t-test. Datasets that did not pass the Pearson and D'Agostino omnibus test were analyzed using a two sided Mann-Whitney U test.

Primers

TYKRIL:
Forward primer sequence
5′-3′: CACCTGCCTGGGAAGTTTCA
Backward primer sequence
5′-3′: ATCTGGATCTGTGTGGTGCC

Further primer sequences are available upon request.

Proximity Ligation Assay

The assay was performed as recommended by the manufacturer (DU092101 SIGMA, Duolink® In Situ Red Starter Kit Mouse/Rabbit from Sigma). In brief cells seeded in 12 Well chamber slides and treated with LNA GapmeRs as described earlier. For doxorubicin (Doxo) treatment, cells were incubated with 1 μg/ml Doxorubicin for 24 h ahead of staining procedure. After LNA transfection or Doxo treatment, cells were washed with ice cold PBS, fixed in ice cold acetone for 10 minutes. After washing and blocking for 30 minutes, primary antibodies (p53: Abcam #ab179477 rabbit 1:500; p300 from ActivMotic #61401, mouse, 1:2000) were incubated 2 hours at 37° C. Subsequently PLA probes (1:5) were added and incubated for 1 h at 37° C. Subsequently cells were washed 2 times and the ligation reagent (1:5) was incubated for 30 minutes at 37° C. followed by amplification with polymerase solution (Sigma). Finally Hoechst was incubated for 10 minutes and probes were embedded in fluoromount. Imaging was done with a Leica SP5 confocal, excitation wavelengths were: 405 nm, 488 nm, 553 nm. Z-stacks maximum projections are shown with z step size of 2 μm.

Dual Luciferase Reporter Array:

Activitiy of transcription factors were performed 48 hours upon TYKRIL knockdown with a dual luciferase “Cignal 45-Pathway Reporter Array” from Qiagen according to the manfucaturer's instruction. In brief, TYKRIL was silenced as described previously. 48 hours after knockdown, cells were seeded at a density of 4×10⁴ cells per well in a cignal 45-Pathway Reporter Array plate and incubated Lipofectamine RNAimax at 37° C. 5% CO2 for 4 hours. Subsequently Medium was switched to pericyte growth medium. 24 hours thereafter luciferase activity from firefly and renilla luciferase were measured in a promega GloMax MultiDetection system.

Endogenous TYKRIL Overexpression RNA Guided Gene Activation

Human pericytes were transduced with a lentivirus pHAGE Ef1alpha dCAS9-VP64 (Addgene #50918) carrying a puromycin selection marker and HA tag. After transduction, successfully transduced pericytes were selected by puromycin treatment (10 mg/ml). For confirmation of successful transduction, immunoblotting against HA and CAS9 was carried out in protein samples in order to verify successful transduction. hPC expressing dCAS9-VP64 were subsequently transfected with guide RNA blocks directed against the TYKRIL promoter region. RNA and protein samples for TYKRIL and PDGFRβ analyses were collected 48 hours after gRNA block transfection. Sequences of gRNA block mixes and primers are available upon request.

Cross Linking RNA Immunoprecipitation

P53 Immunoprecipitation from pericyte protein lysates was carried out using p53 beads (p53-trap Chromotek #pta-20-kit). As negative control GFP beads were used (GFP-trap from Chromotek). In brief pericytes were washed, snap frozen and lysed. Protein lysates were incubated with p53 or GFP beads as recommended by the manufacturer. Following IP, beads were resuspended in 20 μl Proteinase K buffer, incubated at 55° C. for 30 minutes. Subsequently probes were centrifuged for about 60 seconds for 1000 g at 4° C. After centrifugation RNA was isolated using Qiazol as described previously (700 μl Qiazol from Qiagen MiniRNA Kit).

RNA was reversely transcribed and TYKRIL was measured as stated before by realtime PCR.

Ahead of protein isolation, pericytes were exposed towards UV-C light in order to covalently link protein bound RNA.

Primer Sequence for murine TYKRIL:
Forward:
(SEQ ID NO: 7)
AATAAAGCAGTGGGTGCTGGG
Reverse:
(SEQ ID NO: 8)
ACTGTTGCAACCCATTTATCTGA
Sequences of murine LNA Gapmers:
mLNA#1:
(SEQ ID NO: 9)
GGCACACGAACAGCTG
mLNA#2:
(SEQ ID NO: 10)
TGGCACACGAACAGCT
mLNA#3:
(SEQ ID NO: 11)
TGTCTGCACTTAATTA
mLNA#4:
(SEQ ID NO: 12)
GTCTGCACTTAATTAA

Murine TYKRIL Knockout In Vivo:

HPLC purified. LNA GapmeRs were injected intraperitoneally at a dosage of 20 mg/kg bodyweight. 48 hours after injection, mice were sacrificed by isoflurane overdose and cardially perfused with PBS at a steady flow of 9 ml/min. Subsequently organs were removed and snap forzen for RNA and protein isolation.

### Example 1: Characterization of Human Pericytes

In order to validate human PC used in the present study, the inventors evaluated the expression of several established PC markers on protein level. Immunofluorescence revealed a robust expression of PDGFRβ (FIG. 2A), α SMA and NG2 in PC. Immunoblotting of PC lysates for PDGFRβ, NG2, Desmin and αSMA (FIG. 2B) as well as quantitative real time PCR (qRT-PCR) further corroborated the immunofluorescence findings. Counterstains against the endothelial marker von Willebrand factor did not show any significant amounts of contamination of PC cultures with endothelial cells. Another hallmark to identify PC is their ability to form intercellular junctions with endothelial cells. Live cell imaging in coculture assays between PC and HUVEC revealed intercellular dye transfer between both cell types, indicating the exchange of cytoplasmic fractions between HUVEC and PC (FIG. 2C).

### Example 2: Identification and Characterization of the Hypoxia Regulated lncRNA TYKRIL in Human Pericytes

To identify pathologically relevant lncRNAs in PC, the inventors subjected PC towards atmospheric hypoxia to mimic cardiovascular ischemia and tumor hypoxia. Cells were exposed towards 1% O2 and 5% CO2 for 24 h in a humidified atmosphere. Hypoxia resulted in a significant drop of pO2 in cell culture medium (FIG. 1A). In addition, hypoxia induced upregulation of the prototypic hypoxia response gene VEGFA (FIG. 1B) and increased HIF-1α expression as shown by immunoblotting (FIG. 1C). Ischemia resulted in a sparse rate of cell death of about 3 percent in human PC (FIGS. 1D and E), indicating that the majority of PC survived in our experimental hypoxia setting. Deep sequencing analyses identified 30 significantly (n=3; P<0.05) regulated lncRNAs in PC. A heatmap depicts a selection of the most significantly regulated lncRNAs that includes TYKRIL (FIG. 2D). Upregulation of TYKRIL upon hypoxia was verified by qRT-PCR (FIG. 2E). In order to determine the subcellular localization of TYKRIL, the inventors performed qRT-PCR in cytosolic and nuclear fractions under normoxic and hypoxic conditions. Here, the inventors found that TYKRIL is present in both cellular compartments, with a trend to localize into the nucleus under hypoxia (FIG. 2F). Panel in FIG. 2 G depicts the estimated secondary structure of TYKRIL (source: lncipedia.org). TYKRIL is a long intergenic noncoding RNA, flanked by the coding genes CRYAA (upstream) and SIK-1 (downstream) and is localized on chromosome 21:44778027-44782229 next to transcript ENST00000435702 (FIG. 2 H). Data regarding FKPM coverage from the inventors RNAseq data indicates a high coverage of the exons 1 and 2 under hypoxia, whilst up- and downstream coverage of neighbouring sequences are sparse in FKPM readings.

### Example 3: TYKRIL Knockdown by LNA GapmeRs

In order to study the biological function of TYKRIL, the inventors silenced TYKRIL using a locked nucleid acid GapmeR strategy. Locked nucleid acids flanking TYKRIL antisense sequences were designed (purchased from Exiqon). Binding of LNA GapmeRs on TYKRIL induces RNAse H digestion (FIG. 3A), which resulted in a significant knockdown of TYKRIL expression levels in PCs (FIG. 3B). To strengthen the biological significance of TYKRIL knockdown, 2 different LNA GapmeR sequences (LNA#1 and LNA#3) were used to silence the target in order to minimize possible off-target effects of the LNA GapmeRs. Both sequences lead to a significant reduction of TYKRIL.

### Example 4: TYKRIL Silencing Downregulates PDGFRβ Expression on Protein and mRNA Level

Since the inventors observed a significant upregulation of PDGFRβ on mRNA and protein level upon hypoxia (FIG. 4A, B), the inventors were interested in the question if the hypoxia induced lncRNA TYKRIL has an effect of PDGFRβ expression. Silencing of TYKRIL by LNA GapmeRs resulted in a decrease of PDGFRβ mRNA (FIG. 4C), as well as PDGFRβ protein levels (FIG. 4D). PDGFRβ is well known to be pivotal for pericyte function, cell survival and proliferation. It is further well documented that tyrosine kinase inhibition by imatinib induces PC loss. Imatinib is an unselective kinase inhibitor that acts on PDGF receptors such as the stem cell receptor Abl and Kit25 and is used clinically in e.g. cancer treatment. The inventors were therefore interested in the question, if specific PDGFRβ downregulation by TYKRIL silencing may potentiate efficacy of imatinib in vitro. The inventors found that PC became more susceptible towards chemotherapeutic treatment towards imatinib upon TYKRIL knockdown (FIG. 4E). Imatinib treatment alone reduced cell viability about 20%, whilst TYKRIL silencing boosted this effect resulting in a reduction of cell viability of roughly 45% Interestingly, loss of PDGFRβ upon TYKRIL knockdown reduced PC cell numbers (FIG. 4F) by inhibiting cell proliferation as shown by diminishment in Ki67 proliferation indices (FIG. 4G). Moreover the inventors detected a sparse increase in cell death upon and TYKRIL silencing vs. scramble Ctrl (FIG. 1F).

### Example 5: TYKRIL Knockdown Impairs Downstream PDGFRβ Signal Transduction

In order to study the effect of TYKRIL silencing on PDGFRβ downstream signaling the inventors performed PDGF stimulation experiments. In order to achieve selective PDGFRβ stimulation the inventors pretreated PC with PDGF-AA as describe before. An established downstream signaling pathway of PDGFRβ that controls cellular functions such as cell proliferation is AKT2. As expected, AKT phosphorylation is markedly reduced in PC that were treated with TYKRIL LNA Gapmers as indicated by immunoblotting (FIG. 5). These results illustrate that a loss of TYKRIL results in a loss PDGFRβ and related downstream signaling transduction.

### Example 6: TYKRIL is Essential for PC Recruitment Towards Endothelial Cells

PDGFRβ signaling is essential for recruitment of PCs towards endothelial cells. To evaluate if TYKRIL has an impact on PC recruitment, the inventors performed Matrigel coculture assays upon TYKRIL knockdown. Here, it was found that TYKRIL silencing significantly impaired PC recruitment towards HUVEC (FIG. 6) compared with PC treated with LNA control sequences.

Here the inventors demonstrate that hypoxia triggers a significant change in lncRNA expression in human PC. Moreover the inventors show that the hypoxia induced long noncoding RNA TYKRIL is a pro-angiogenic lncRNA, that is essential for proper human PC function by stabilizing PC proliferation, PC recruitment and prevention of PC cell death through induction of PDGFRβ expression.

The main findings of this invention are: i) TYKRIL is induced upon hypoxia in pericytes and is expressed in the cytosol as well as in the nucleus of PC. ii) TYKRIL can be effectively silenced by LNA GapmeRs, which results in a significant downregulation of the tyrosine kinase receptor PDGFRβ on mRNA and protein level. iii) Loss of PDGFRβ upon TYKRIL silencing results in a decrease of PC proliferation, increases PC cell death, enhances susceptibility towards chemotherapeutic treatment and impairs PC recruitment towards endothelial cells.

Various studies have shown that targeting PDGFRβ signaling by genetic ablation or by pharmacological inhibition induces a loss of PC, that goes along with vascular malfunction which is capable to reduce tumor growth in a mouse lymphoma model (Ruan, J. et al. Imatinib disrupts lymphoma angiogenesis by targeting vascular pericytes. Blood 121, 5192-5202 (2013). Moreover clinical trials have shown that targeting PC or PDGFβ by chemotherapy improves clinical outcome by inhibition of neovascularization of tumors (Apperley, J. F. et al. Response to imatinib mesylate in patients with chronic myeloproliferative diseases with rearrangements of the platelet-derived growth factor receptor beta. N. Engl. J. Med. 347, 481-487 (2002)). Therefore, TYKRIL is a new therapeutic target to impair cancer angiogenesis, and TKRIL inhibitors as described herein are useful new medicines for treating various proliferative diseases as described herein above.

PDGFRβ signaling is initiated upon binding of the peptide PDGF-BB, a potent ligand to PDGFRα and PDGFRβ, which is secreted by various cell types including endothelial cells, several tumour cell lines and platelets. PDGF-BB stimulation leads to dimerization of the intracellular PDGFRβ domain inducing autophosphorylation of various tyrosine residues that activate several signaling pathways that include e.g. Ras, PI3-Kinase and PLC-γ. It is well documented that intact PDGFRβ is essential for cell proliferation and mediates the recruitment of PC towards endothelial cells (Tallquist, M. D., French, W. J. & Soriano, P. Additive effects of PDGF receptor beta signaling pathways in vascular smooth muscle cell development. PLoS Biol. 1, E52 (2003)), thereby promoting vessel maturation and stabilizing endothelial barrier function.

Interestingly the inventors also found that silencing of TYKRIL further increased the susceptibility of PC towards pharmacologic PDGFRβ inhibition by imatinib. AKT signaling has been shown to be pivotal for vessel maturation and stability. In line with the data show here, Chen et al. have shown that a loss of AKT affects vessel maturation. In this study the inventors observed that PC lacking TYKRIL, pericyte recruitment towards endothelial cells, a hallmark for vessel maturation, is significantly impaired that is likely due to a decrease in AKT phosphorylation. Hence TYKRIL acts together with imatinib synergistically, which is surprising. TYKRIL therefore will help to overcome cancer resistancy e.g. in lymphoma treatment by boosting efficacy of imatinib. It is apparent to those of skill in the art that the sysnergistic effect of TYKRIL towards imatinib is transferable to other tyrosine kinase inhibitors, specifically other multi kinase inhibitors as mentioned herein above, or PDGFRβ inhibitors.

Since the inventors found a decrease of PDGFRβ expression on mRNA and protein level, the inventors suggest that TYKRIL exerts its function by reducing the transcription of PDGFRβ or by degrading PDGFRβ mRNA. The inventor's findings are of therapeutic relevance in a clinical setting. TYKRIL was specifically upregulated under hypoxic conditions, which are also present in malignant or angiogenic processes and cardiovascular ischemia (Zehendner, C. M. et al. Moderate Hypoxia Followed by Reoxygenation Results in Blood-Brain Barrier Breakdown via Oxidative Stress-Dependent Tight-Junction Protein Disruption. PLoS ONE 8, e82823 (2013)). Moreover the inventors demonstrate significant regulation of TYKRIL and TYKRIL-PDGFRβ interdependence in disease states such as i) Heart failure, ii) PAH and iii) glioblastoma. Hence, silencing of TYKRIL represents an effective strategy to block angiogenesis in cancer (e.g. glioblastoma) or to prevent organ remodeling in PAH, stroke or myocardial infarction.

### Example 7: TYKRIL Modulates PDGFRβ Expression and Acts as a Suppressor of the Tumor Antigen p53

It is known that lncRNAs may exert their function by regulating transcription factor activity in the nucleus. TYKRIL knockdown resulted in perciyte de-differentiation and loss of PDGFRβ as indicated by RNA seq, immunoblotting and qPCR (FIG. 7 A-C). Since TYKRIL localized partly in the nucleus (FIG. 7 D) transcription factor array profiling analyses was performed to evaluate if a loss of TYKRIL has an effect on transcription factor activity. Here it was found that the tumor suppressor p53 is most prominently upregulated after TYKRIL loss (FIG. 7 E). RNA seq in TYKRIL knockdown demonstrate a significant inverse regulation of TYKRIL and p53 and p53 dependent genes (FIGS. 7 F and G). Endogenous p53 activation by doxorubicin treatment resulted in a significant decline in PDGFRβ expression and TYKRIL downregulation (FIG. 7 H-J). Co-silencing p53 and TYKRIL resulted in a complete rescue with regard to cell viability loss upon TYKRIL knockdown (FIG. 7 L). These data point towards a regulatory feedback loop between TYKRIL and p53 which regulates the PDGFRβ (FIG. 7L). Sequence of LNA#2 corresponds to LNA#3 in FIGS. 1-6.

In order to study TYKRIL gain of function human primary pericytes constitutively expressing CAS9 mutant carrying the transcriptional activator domain VP64 which enables RNA guided gene activation5 (FIG. 8 A, B) was established. RNA guided gene activation of TYKRIL resulted in a significant upregulation of TYKRIL and PDGFRβ (FIG. 8 C, D). Importantly, LNA GapmeR cotransfection with gRNAs blunted TYKRIL upregulation and decreased PDGFRβ overexpression (FIG. 8 E, F), demonstrating specificity of LNA GapmeRs. Sequence of LNA#2 corresponds to LNA#3 in FIGS. 1-6.

### Example 8: TYKRIL Physically Interacts with p53 Thereby Preventing the Binding of the p53 Co-Activator p300

To further dissect how TYKRIL modulates p53 activity, cross linking RNA Immunoprecipitation experiments were performed. It was found that TYKRIL directly interacts with the tumor antigen p53 (FIG. 9). p53 is tightly regulated by post-translational modifications such as acetylation and phosphorylation. Thereby, co-activators such as the acetyltransferase p300 lead to p53 acetylation and translocation of the p53-p300 complex into the nucleus, whilst the ubiquitin ligase MDM2 rapidly degrades p53. Proximity ligation assays (PLA) allow to precisely quantify and visualize protein-protein interactions. PLA imaging upon TYKRIL knockdown demonstrate a significant increase of p53-p300 interaction upon TYKRIL loss (FIG. 10). These results illustrate that TYKRIL prevents p53-p300 interaction by directly binding to p53, thereby blocking the direct interaction with the p53 co-activator p300 and subsequent nuclearization of the protein complex. Sequence of LNA#2 corresponds to LNA#3 in FIGS. 1-6.

### Example 9: TYKRIL and PDGFRβ Expression Significantly Correlate in Pulmonary Arterial Hypertension, Heart Failure and Glioblastoma Multiforme

In order to show TYKRIL-PDGFRβ interdependence in human disease, TYKRIL was measured in the myocardium of patients diagnosed with heart failure (FIG. 11 A). Heart failure is associated with a significantly impaired microcirculation. Here, it was found that TYKRIL and PDGFRβ were significantly reduced compared to myocardial specimens from patients without the diagnosis of heart failure (FIG. 11 B, C). In addition, it was found that there is a significant positive correlation between TYKRIL and PDGFRβ in heart failure disease (FIG. 11 D). Interestingly lncRNA RP11-65J21.3 was also found to be significantly reduced in heart failure (0.52 fold change versus control, n=18 HF heart failure patients, p<0.05). In summary these data confirm TYKRIL-PDGFRβ interdependence in human cardiac disease.

Pulmonary arterial hypertension is a disease state that is partially attributed to the abnormal growth of contractile cells that narrow the lumen of microvessels in the lung which enhances pulmonary artery resistance. Since pericytes are known to display contractile characteristics, TYKRIL and PDGFRβ were measured in lung tissue from patients diagnosed with PAH and a control cohort (FIG. 11 E). Interestingly, it was found that TYKRIL and PDGFRβ significantly correlated with each other (FIG. 11 F), indicating that enhanced TYKRIL expression goes along with enhanced PDGFRβ expression in health and lung disease. TYKRIL therefore represents a promising molecular target to modulate PDGFRβ expression in the pulmonary system because abnormal PDGFRβ expression is known to play a role in lung diseases such COPD, fibrosis, lung emphysema (Tomasovic, A. et al. Sestrin 2 Protein Regulates Platelet-derived Growth Factor Receptor β (Pdgfrβ) Expression by Modulating Proteasomal and Nrf2 Transcription Factor Functions. J. Biol. Chem. 290, 9738-9752 (2015) and Rowley, J. E. & Johnson, J. R. Pericytes in Chronic Lung Disease. Int. Arch. Allergy Immunol. 164, 178-188 (2014)).

Glioblastoma multiforme is a malignant brain tumor with abnormal angiogenesis, poor prognosis and few treatment options. This is due to the inefficacy of chemotherapy and early tumor relapse following resection since glioblastoma stem cells are capable of generating pericytes that facilitate revascularization of the glioblastoma tissue. RNA Seq analyses from 39 glioblastoma core regions compared with n=19 brain resections from patients diagnosed with epilepsy (FIG. 11 G) revealed a significant upregulation of TYKRIL and PDGFRβ in glioblastoma multiforme (FIG. 11 H, I). Based on these data TYKRIL fosters uncontrolled tumor angiogenesis by stabilizing PDGFRβ expression. Interestingly, p53 was significantly enhanced in the tumor cohort (FIG. 11 J), pointing towards an uncoupling of the TYKRIL-p53 feedback loop under physiological conditions.

### Example 10: Single Shot Administration of Anti-TYKRIL LNA GapmeRs Induces TYKRIL Downregulation and Loss of PDGFRβ In Vivo

In order to demonstrate in vivo relevance of TYKRIL signaling the murine TYKRIL orthologue in locus conservation was identified. PCR from RNA isolated from primary mouse pericytes under normoxic and hypoxic conditions with primers directed the genomic locus chr17:31805539-31805608 (GRCm38/mm10) (FIG. 12 A) demonstrate the presence of a previously unknown, hypoxia regulated murine transcript which is present: +/−3 kbp from locus chr17:31805539-31805608 (GRCm38/mm10) (FIG. 12 A). Silencing murine TYKRIL with various mLNA GapmeRs resulted in a downregulation of PDGFRβ (FIG. 12 B). mLNA#4 was used for further in vivo experiments in order to demonstrate relevance of TYKRIL signaling in vivo. The murine TYKRIL orthologue in the mouse was silenced by intraperitoneal single shot injection of LNA GapmeRs (FIG. 13 A). Here, a downregulation of TYKRIL and the PDGFRβ in heart (FIG. 13 B, C), lungs (FIG. 13 D), liver (FIG. 13 E), spleen (FIG. 13 F) and kidneys (FIG. 13 G) was found. These results indicate that TYKRIL can be sufficiently targeted in vivo in all major organ systems except healthy CNS due to the blood brain barrier selectivity. Importantly, identification of the murine TYKRIL orthologue will allow screening for the importance of TYKRIL signaling in all available mouse in vivo models mimicking human disease.

1. A method of treating a disease in a subject, comprising the step of administering an inhibitor of a long non-coding RNA (lncRNA) selected from TYKRIL, MIR210HG, RP11-367F23.1, H19, RP11-44N21.1, AC006273.7, RP11-120D5.1, AP001046.5, RP11-443B7.1, AC005082.12, RP11-65J21.3, or AC008746.12, to the subject.

2. The method of claim 1, wherein the lncRNA is Tyrosine Kinas Receptor Inducing lncRNA (TYKRIL) and has a sequence of at least 80% sequence identity to SEQ ID NO: 1.

3. The method of claim 1, wherein the inhibitor is a lncRNA antisense molecule, or an antisense expression molecule, or small molecule inhibitors, RNA/DNA-binding proteins/peptides, or an anti-lncRNA antibody.

4. The method of claim 3, wherein the lncRNA antisense molecule is a nucleic acid oligomer having a contiguous nucleotide sequence of a total of 8 to 100 nucleotides, wherein said contiguous nucleotide sequence is at least 80% identical to the reverse complement of the sequence of the lncRNA.

5. The method of claim 3, wherein the lncRNA antisense molecule comprises contiguous nucleotide sequence having at least one nucleic acid modification, preferably selected from 2′-0-alkyl modifications, such as 2′-0-methoxy-ethyl (MOE) or 2′-0-Methyl (OMe), ethylene-bridged nucleic acids (ENA), peptide nucleic acid (PNA), 2′-fluoro (2′-F) nucleic acids such as 2′-fluoro N3-P5′-phosphoramidites, 1′, 5′-anhydrohexitol nucleic acids (FINAs), and locked nucleic acid (LNA).

6. The method of claim 1, wherein the disease in the subject administered the inhibitor of the long non-coding RNA (lnc RNA) is associated with an increased expression of a Platelet-derived growth factor receptor (PDGFR) and/or associated with a decreased expression/function of p53, an eye disease, fibrotic disease (fibrosis), vascular disease and/or a tumorous disease.

7. The method of any of claim 1, further comprising the step of administering, simultaneously or sequentially with the inhibitor of the lncRNA, a second therapeutic agent.

8. The method of claim 17, wherein the PDGFR-inhibitor is an anti-PDGFR-antibody, a small molecule tyrosine kinase inhibitor, preferably imatinib, sorafenib, lapatinib, BIRB-796 and AZD-1152; AMG706, Zactima (ZD6474), MP-412, sorafenib (BAY 43-9006), dasatinib, CEP-701 (lestaurtinib), XL647, XL999, Tykerb (lapatinib), MLN518, PKC412, STI571, AEE 788, OSI-930, OSI-817, Sutent (sunitinib maleate), axitinib (AG-013736), erlotinib, gefitinib, axitinib, temsirolimus and nilotinib (AMN107).

9. A medicinal combination comprising
a) an inhibitor of a lncRNA selected from TYKRIL, MIR210HG, RP11-367F23.1, H19, RP11-44N21.1, AC006273.7, RP11-120D5.1, AP001046.5, RP11-443B7.1, AC005082.12, RP11-65J21.3, or AC008746.12, and
b) a PDGFR inhibitor.

10. (canceled)

11. The method of claim 1, further including the step of administering a pharmaceutical acceptable carrier and/or excipient to the subject.

12. An in-vitro method for screening a modulator of the expression and/or function of a lncRNA selected from selected from TYKRIL, MIR210HG, RP11-367F23.1, H19, RP11-44N21.1, AC006273.7, RP11-120D5.1, AP001046.5, RP11-443B7.1, AC005082.12, RP11-65J21.3, or AC008746.12, comprising the steps of,
a) Providing a sample of pericytes,
b) Optionally, inducing hypoxia in the sample of pericytes,
c) Contacting the sample of pericytes with a candidate compound,
d) Determining at least one of the following in the sample of pericytes:
i) An expression level of the lncRNA,
ii) An expression level of PDGFR,
iii) recruitment of the pericytes towards endothelial cells,
iv) proliferation of pericytes,
v) activity or expression of p53, or
vi) interaction of p53 with the histone acetyltransferase p300,
wherein a significant change in any of (i) to (vi) compared to a control indicates that the candidate compound is a modulator of the lncRNA expression and/or function of the lncRNA.

13. The method according to claim 12, wherein a reduced expression in (i) and/or (ii) compared to a control, and/or an impaired recruitment in (iii) compared to a control, and/or a reduced proliferation in (iv), and/or altered expression or activity of p53 in (v), and/or altered interaction of p53 with its co-activator p300 in (vi) indicates that the candidate compound is an inhibitor of lncRNA expression and/or function.

14. The method according to claim 12, wherein step (b) and step (c) may be performed in reverse order or simultaneously.

15. The method according to claim 12, wherein the modulator of the expression and/or function of the lncRNA identifies an inhibitor TYKRIL.

16. The method of claim 3, wherein the lncRNA antisense molecule is antisense RNA, RNA interference (RNAi), siRNA, esiRNA, shRNA, miRNA, decoys, RNA aptamers, GapmeRs, or LNA molecules.

17. The method of claim 6, wherein the PDGFR is a PDGFR-β.

18. The method of claim 6, wherein the vascular disease is leukemia.

19. The method of claim 7, wherein the therapeutic agent administered to the subject is a PDGFR-inhibitor.

20. The method of claim 19, further including the step of administering a pharmaceutical acceptable carrier and/or excipient to the subject.
